SEQUENCE LISTING (6 sequences)

SEQ ID NO: 1 - protein, 5 aa, Artificial Sequence
  Description of Artificial Sequence Synthetic peptide
GGNCC

SEQ ID NO: 2 - protein, 5 aa, Artificial Sequence
  Description of Artificial Sequence Synthetic peptide
GNNCC

SEQ ID NO: 3 - protein, 5 aa, Artificial Sequence
  Description of Artificial Sequence Synthetic peptide
GGKCC

SEQ ID NO: 4 - protein, 5 aa, Artificial Sequence
  Description of Artificial Sequence Synthetic peptide
GGHCC

SEQ ID NO: 5 - protein, 8 aa, Artificial Sequence
  Description of Artificial Sequence Synthetic peptide
RRNNCCIQ

SEQ ID NO: 6 - protein, 5 aa, Artificial Sequence
  Description of Artificial Sequence Synthetic peptide
GNCCG

(19) United States
(12) Patent Application Publication
(10) Pub. No.: US 2014/0037626 A1
(43) Pub. Date: Feb. 6, 2014

(54) Metal Abstraction Peptide With Release of Metal

(71) Applicants: Echogen, Inc., Lenexa, KS (US); University of Kansas, Lawrence, KS (US)

(72) Inventors: Jennifer Ann Stowell Laurence, Lawrence, KS (US); Mary Elizabeth Krause, Lawrence, KS (US); George Laurence, Lawrence, KS (US)

(73) Assignees: Echogen, Inc., Lenexa, KS (US); University of Kansas, Lawence, KS (US)

(21) Appl. No.: 13/945,769

(22) Filed: Jul. 18, 2013

Related U.S. Application Data
(60) Provisional application No. 61/672,906, filed on Jul. 18, 2012.

(51) Int. Cl. C07K 7/06

(52) U.S. Cl. CPC C07K 7/06
USPC 424/134.1; 424/649

(57) ABSTRACT

Compositions comprising peptides that are capable of binding a metal in a square planar orientation, a square pyramidal orientation, or both, are disclosed. Such compositions can be used for binding and releasing a metal in a variety of contexts and environments, such as the treatment of cancer.

### CROSS REFERENCE

This application claims the benefit of U.S. Provisional Application No. 61/672,906, filed on Jul. 18, 2012, the contents of which is incorporated by reference herein in its entirety.

### BACKGROUND OF THE INVENTION

Tags that can be encoded in the genetic material of an organism for recombinant expression of proteins have been utilized for purification and identification of protein products. The advantage of a peptide tag is that the tag is covalently attached to the protein of interest without the need for additional chemical steps to label the protein. One example of such a tag is a poly His-tag, which provides a means of isolating the tagged protein from whole cells using immobilized metal affinity chromatography (IMAC). Other peptide-based tags have been developed to allow for detecting a tagged protein in cell culture assays or cell lysates using antibodies that recognize the peptide tag. While these technologies might be useful in in situ or in vitro assays, they generally are not applicable to in vivo analysis. Moreover, such peptide tags have limited or no functionality outside of protein purification or identification.

### SUMMARY OF THE INVENTION

In some embodiments, the invention provides a pharmaceutical composition comprising: i) a metal-binding peptide, wherein the metal-binding peptide comprises a sequence Z¹—XZ³C₁Z⁴C₂—Z², wherein X is any natural or non-natural amino acid or amino acid analogue, and wherein C₁ and C₂ are each individually chosen from a cysteine and a sulfur-containing alpha or beta amino acid, and wherein Z¹, Z², Z³, and Z⁴ are each individually a sequence of 1-5 residues, or absent, wherein each residue is independently a natural or non-natural amino acid or analogue thereof; ii) a metal bound to the metal-binding peptide; and iii) a pharmaceutically-acceptable excipient, wherein the composition is a unit dosage form.

In some embodiments, the invention provides a method of treating cancer, the method comprising administering to a subject in need or want thereof a therapeutically-effective amount of a composition comprising: i) a metal-binding peptide, wherein the metal-binding peptide comprises a sequence Z¹—XZ³C₁Z⁴C₂—Z², wherein X is any natural or non-natural amino acid or amino acid analogue, and wherein C₁ and C₂ are each individually chosen from a cysteine and a sulfur-containing alpha or beta amino acid, and wherein Z¹, Z², Z³, and Z⁴ are each individually a sequence of 1-5 residues, or absent, wherein each residue is independently a natural or non-natural amino acid or analogue thereof; ii) a metal bound to the metal-binding peptide; and iii) a pharmaceutically-acceptable excipient, wherein the composition is a unit dosage form.

In some embodiments, the invention provides a method of providing a metal to a subject in need or want thereof, the method comprising administering to the subject: i) a metal-binding peptide, wherein the metal-binding peptide comprises a sequence Z¹—XZ³C₁Z⁴C₂—Z², wherein X is any natural or non-natural amino acid or amino acid analog, and wherein C₁ and C₂ are each individually chosen from a cysteine and a sulfur-containing alpha or beta amino acid, and wherein Z¹, Z², Z³, and Z⁴ are each individually a sequence of 1-5 residues, or absent, wherein each residue is independently a natural or non-natural amino acid or analogue thereof; and ii) the metal, wherein the metal is bound to the metal-binding peptide, wherein the metal-binding peptide releases the metal in a physiological environment, wherein the release is associated with a decrease in pH.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 panel A shows incorporation of Ni into the NCC sequence, as observed by absorption spectroscopy. Panel B shows incorporation of Pt into the NCC sequence, as observed by absorption spectroscopy.

FIG. 2 shows CD spectra of Ni-NCC and Ni-GGNCC (SEQ ID NO.:1).

FIG. 3 panel A shows mass spectra collected for Ni-NCC. Panel B shows mass spectra collected for Pt-NCC.

FIG. 4 panel A shows isothermal titration calorimetry (ITC) data for Ni-GGNCC (SEQ ID NO.:1). Panel B shows isothermal titration calorimetry (ITC) data for Pt-GGNCC (SEQ ID NO.:1).

FIG. 5 panel A illustrates a possible arrangement for the binding of a NCC to nickel in which the two sulfur ligands are arranged cis, one nitrogen ligand is derived from a backbone amide, and the second is from the N-terminal amine. Panel B illustrates a possible arrangement for the binding of a GGNCC (SEQ ID NO.:1) peptide to nickel.

FIG. 6 shows metals that bind to the MAP tag, as characterized by absorption spectroscopy.

FIG. 7 shows absorbance profiles revealing the pH-dependent release of metal from GNNCC (SEQ ID NO.:2).

FIG. 8 panel A shows MS of Ni-NCC at high pH, and panel B shows MS of Ni-NCC and the release of the metal after a decrease in the pH.

FIG. 9 shows that pH-dependent release of nickel from GNNCC (SEQ ID NO.: 2) occurs rapidly between pH 6-7.

FIG. 10 shows Pt-NCC in phosphate buffer with excess ammonium chloride at neutral and low pH. The slight increase in intensity at >300 nm at pH 2.47 is due to light scattering from precipitation.

FIG. 11 shows: 1) Pt-NTA (method of Pt transfer to the peptide complex); 2) Pt-GGNCC (SEQ ID NO.: 1) at low pH; 3) Pt-GGNCC (SEQ ID NO.: 1); and 4) ¹H NMR of GGNCC (SEQ ID NO.: 1).

FIG. 12 shows MS of Pt-NCC after exposure to low pH conditions in the presence of DMSO. The m/z of 339.13 corresponds to the mass of free peptide (NCC).

FIG. 13 shows UV absorption assay measuring pH-dependent release of Pt from the NCC tripeptide. Percent complex remaining is calculated from the residual intensity of a characteristic spectral feature at 315 nm.

FIG. 14 shows pH-dependent release of cobalt from NCC.

FIG. 15 panel A shows CD spectra of Ni-tripeptide complexes. Panel B shows the CD spectra of Ni-pentapeptide complexes (SEQ ID NO.: 1, SEQ ID NO.: 3, and SEQ ID NO.: 4).

FIG. 16 shows pH-dependent metal release as shown by absorption scans at different pH values for six different Ni-peptide complexes. Panel A shows the pH-dependent metal release of Ni-GGHCC (SEQ ID NO.: 4) complexes. Panel B shows the pH-dependent metal release of Ni-GGKCC (SEQ ID NO.: 3) complexes. Panel C shows the pH-dependent metal release of Ni-GNNCC (SEQ ID NO.: 2) complexes. Panel D shows the pH-dependent metal release of Ni-HCC complexes. Panel E shows the pH-dependent metal release of Ni-KCC complexes. Panel F shows the pH-dependent metal release of Ni-NCC complexes.

FIG. 17 panel A shows pH-dependent metal release curves for Ni-tripeptide complexes. Panel B shows pH-dependent metal release curves for Ni-pentapeptide complexes.

FIG. 18 shows pH-dependent nickel release curve for PRL-1.

### DETAILED DESCRIPTION

The present disclosure generally relates to metal abstraction peptide tags (MAP tags) and to methods of preparing and using such tags for a variety of uses.

As used herein, the abbreviations for the natural L-enantiomeric amino acids are conventional and are as follows: alanine (A, Ala); arginine (R, Arg); asparagine (N, Asn); aspartic acid (D, Asp); cysteine (C, Cys); glutamic acid (E, Glu); glutamine (Q, Gln); glycine (G, Gly); histidine (H, His); isoleucine (I, Ile); leucine (L, Leu); lysine (K, Lys); methionine (M, Met); phenylalanine (F, Phe); proline (P, Pro); serine (S, Ser); threonine (T, Thr); tryptophan (W, Trp); tyrosine (Y, Tyr); valine (V, Val). Typically, Xaa can indicate any amino acid. In some embodiments, X can be asparagine (N), glutamine (Q), histidine (H), lysine (K), or arginine (R).

Some embodiments of the invention contemplate D-amino acid residues of any standard or non-standard amino acid or analogue thereof.

When an amino acid sequence is represented as a series of three-letter or one-letter amino acid abbreviations, the left-hand direction is the amino terminal direction and the right-hand direction is the carboxy terminal direction, in accordance with standard usage and convention.

The term “metal” as used herein refers to metals in elemental form, metal atoms, and metal ions interchangeably. The term “metal” also encompasses metal radioisotopes.

### Compositions and Methods of the Invention.

The present invention provides compositions for use in treating cancer. Such compositions comprise a metal-binding peptide and a metal bound to the metal-binding peptide. In various embodiments, the metal is a radioisotope. The compositions further comprise a pharmaceutically-acceptable excipient. In some embodiments, the compositions further comprise buffer. In some embodiments, the buffer is a phosphate buffer. In some embodiments, the composition has a pH between about 7 and about 8. In some embodiments, the composition has a pH of about 7.4.

In some embodiments, the composition for treating cancer comprises a metal-binding peptide; a metal bound to the metal-binding peptide; and a pharmaceutically-acceptable excipient, wherein the metal-binding peptide binds the metal with an affinity constant, wherein the affinity constant is at least 2 times greater at a pH above 7 than an affinity constant at a pH below 6. In some embodiments, the affinity constant at higher pH is at least 10 times greater than the affinity constant at lower pH. In some embodiments, the affinity constant at a pH between 7 and 8 is at least 10 times greater than an affinity constant at a pH between 4 and 6.

In some embodiments, the composition for treating cancer comprises a metal-binding peptide; a metal bound to the metal-binding peptide; and a pharmaceutically-acceptable excipient; wherein the composition contains a concentration of metal-binding peptide and a concentration of metal, and wherein the metal-binding peptide binds the metal to yield a concentration of peptide with bound metal and a concentration of peptide without bound metal and a ratio of the concentrations, wherein the ratio is at least two times greater at a pH above 7 than the ratio at a pH below 6 for a constant concentration of metal-binding peptide and metal. In some embodiments, the ratio is at least five times greater at a pH above 7 than the ratio at a pH below 6. In some embodiments, the concentration of metal-binding peptide and the concentration of metal are in a ratio of about 1 to about 1 or greater than about 1 to about 1. In some embodiments, the concentration of metal-binding peptide and the concentration of metal are in a ratio ranging from about 1:about 1 to about 2:about 1. In some embodiments, the ratio is at least five times greater at a pH between 7 and 8 than the ratio at a pH between 4 and 6.

A metal binding peptide can bind to metal in various ratios. In some embodiments, the concentration of metal-binding peptide and the concentration of metal are in a ratio of about 1:about 1; about 1:about 2; about 1:about 3; about 1:about 4; about 1:about 5; about 1:about 6; about 1:about 7; about 1:about 8; about 1:about 9; or about 1:about 10. In some embodiments, the metal-binding peptide can bind to metal in a ratio of about 10:about 1; about 9:about 1; about 8:about 1; about 7:about 1; about 6:about 1; about 5:about 1; about 4:about 1; about 3:about 1; or about 2:about 1. In some embodiments, the ratio is 1:1.

A metal-binding peptide can bind to metal at different pH levels. A metal binding peptide can bind to a metal at a pH level of about 6, about 6.1, about 6.2, about 6.3, about 6.4, about 6.5, about 6.6, about 6.7, about 6.8, about 6.9, about 7, about 7.1, about 7.2, about 7.3, about 7.4, about 7.5, about 7.6, about 7.7, about 7.8, about 7.9, about 8, about 8.1, about 8.2, about 8.3, about 8.4, about 8.5, about 8.6, about 8.7, about 8.8, about 8.9, about 9, about 9.1, about 9.2, about 9.3, about 9.4, about 9.5, about 9.6, about 9.7, about 9.8, about 9.9, or about 10.

A metal-binding peptide can release a metal at a pH of about 7.5, about 7.4, about 7.3, about 7.2, about 7.1, about 7, about 6.9, about 6.8, about 6.7, about 6.6, about 6.5, about 6.4, about 6.3, about 6.2, about 6.1, about 6, about 5.9, about 5.8, about 5.7, about 5.6, about 5.5, about 5.4, about 5.3, about 5.2, about 5.1, about 5, about 4.9, about 4.8, about 4.7, about 4.6, about 4.5, about 4.4, about 4.3, about 4.2, about 4.1, about 4, about 3.9, about 3.8, about 3.7, about 3.6, about 3.5, about 3.4, about 3.3, about 3.2, about 3.1, about 3, about 2.9, about 2.8, about 2.7, about 2.6, about 2.5, about 2.4, about 2.3, about 2.2, about 2.1, or about 2.

In some embodiments, the metal-binding peptide binds to the metal at a pH above 6. In some embodiments, the metal-binding peptide binds to the metal at a pH above 7. In some embodiments the metal-binding peptide releases the metal at a pH below 7. In some embodiments the metal-binding peptide releases the metal at a pH below 6.

In some embodiments, the invention provides for a metal-binding peptide or a radioisotope-binding peptide comprising a sequence XC₁C₂, wherein X is any natural or non-natural amino acid or amino acid analogue, and further wherein C₁ and C₂ are each individually chosen from a cysteine and a sulfur-containing alpha or beta amino acid. In some embodiments, the sequence XC₁C₂ is included in a sequence Z¹—XC₁C₂, wherein Z¹ is any natural or non-natural amino acid or sequence of natural or non-natural amino acids or analogues thereof. In some embodiments, the sequence XC₁C₂ is included in a sequence XC₁C₂—Z², wherein Z² is any natural or non-natural amino acid or sequence of natural or non-natural amino acids or analogues thereof. In some embodiments, the sequence XC₁C₂ is included in a sequence Z¹—XC₁C₂—Z², wherein Z¹ and Z² are each individually any natural or non-natural amino acid or sequence of natural or non-natural amino acids. In some embodiments, at least one of Z¹ and Z² includes a basic amino acid adjacent to either X or C₂.

In some embodiments, the invention provides for a metal-binding peptide or a radioisotope-binding peptide comprising a sequence XZ³C₁C₂, wherein X is any natural or non-natural amino acid or amino acid analogue, and wherein C₁ and C₂ are each individually chosen from a cysteine and a sulfur-containing alpha or beta amino acid, and wherein Z³ is a sequence of 1-5 residues, wherein each residue is independently a natural or non-natural amino acid or analogue thereof. In some embodiments, the metal-binding peptide comprises a sequence XC₁Z⁴C₂, wherein X is any natural or non-natural amino acid or amino acid analogue, and wherein C₁ and C₂ are each individually chosen from a cysteine and a sulfur-containing alpha or beta amino acid, and wherein Z⁴ is a sequence of 1-5 residues, wherein each residue is independently a natural or non-natural amino acid or analogue thereof. In some embodiments, the metal-binding peptide comprises a sequence XZ³C₁Z⁴C₂, wherein X is any natural or non-natural amino acid or amino acid analogue, and wherein C₁ and C₂ are each individually chosen from a cysteine and a sulfur-containing alpha or beta amino acid, and wherein Z³ and Z⁴ are each individually a sequence of 1-5 residues, wherein each residue is independently a natural or non-natural amino acid or analogue thereof. Any of Z¹, Z², Z³, and Z⁴ can be present or absent.

The invention provides for a metal-binding peptide comprising a sequence Z¹—XZ³C₁Z⁴C₂—Z², wherein X is any natural or non-natural amino acid or amino acid analogue, and wherein C₁ and C₂ are each individually chosen from a cysteine and a sulfur-containing alpha or beta amino acid, wherein Z¹ and Z² are each individually any natural or non-natural amino acid or sequence of natural or non-natural amino acids, or absent, and wherein Z³ and Z⁴ are each individually a sequence of 1-5 residues, or absent, wherein each residue is independently a natural or non-natural amino acid or analogue thereof. In some embodiments, at least one of Z¹, Z², Z³, and Z⁴ is not absent. In some embodiments, the sequence is 1-2 residues long, 1-3 residues long, 1-4 residues long, 1-5 residues long, 2-3 residues long, 2-4 residues long, 2-5 residues long, 3-4 residues long, 3-5 residues long, and/or 4-5 residues long.

In some embodiments, at least one of Z¹, Z², Z³, and Z⁴ includes a basic amino acid adjacent to X, C₁, or C₂.

In some embodiments, the metal-binding peptide or radioisotope-binding peptide comprises at least 20 amino acids. The metal-binding peptide can adopt a tertiary structure under physiological conditions. In some embodiments, a basic amino acid located at least 17 amino acids away from C₁ by amino acid sequence is located within 20 angstroms in space from C₁. In some embodiments, the basic amino acid is arginine. In some embodiments, the basic amino acid is lysine. In some embodiments, the basic amino acid is histidine.

In some embodiments, the metal-binding peptide or radioisotope-binding peptide is linked to an antibody or included within an antibody amino acid sequence. The antibody can be linked to or can include more than one such peptide. Linkers include amide or non-amide bond linkages. For example, for an amide bond linker, the link can occur through the terminal nitrogen of a lysine side chain. In some embodiments, the metal-binding peptide is linked to the antibody through a non-amide bond.

In some embodiments, the metal or radioisotope is selected from Group 3 metals, Group 4 metals, Group 5 metals, Group 6 metals, Group 7 metals, Group 8 metals, Group 9 metals, Group 10 metals, Group 11 metals, Group 12 metals, Group 13 metals, Group 14 metals, Group 15 metals, the lanthanide metals, the actinide metals, and the transuranic metals. For example, the metal or radioisotope can be selected from Group 10 metals, Group 11 metals, and Group 13 metals. In some embodiments, the metal is selected from the lanthanide metals, the actinide metals, and the transuranic metals. In some embodiments, the metal is platinum, palladium, gallium, or gadolinium. In some embodiments, the metal is platinum. The radioisotope can be, for example, an alpha emitter, a beta emitter, a positron emitter, and/or a gamma emitter. In some embodiments, the radioisotope is of a transition metal. In some embodiments, the metal or radioisotope is an isotope of any of Scandium, Titanium, Vanadium, Chromium, Manganese, Iron, Cobalt, Nickel, Copper, Zinc, Yttrium, Zirconium, Niobium, Molybdenum, Technetium, Ruthenium, Rhodium, Palladium, Silver, Cadmium, Lutetium, Hafnium, Tantalum, Tungsten, Rhenium, Osmium, Iridium, Platinum, Gold, Mercury, Lawrencium, Rutherfordium, Dubnium, Seaborgium, Bohrium, Hassium, Meitnerium, Darmstadtium, Roentgenium, or Ununbium.

In some embodiments, the radioisotope is of a lanthanide metal. In some embodiments, the metal or radioisotope is an isotope of any of Lanthanum, Cerium, Praseodymium, Neodymium, Promethium, Samarium, Europium, Gadolinium, Terbium, Dysprosium, Holmium, Erbium, Thulium, Ytterbium, or Lutetium.

In some embodiments, the radioisotope is of an actinide metal. In some embodiments, the metal or radioisotope is an isotope of any of Actinium, Thorium, Protactinium, Uranium, Neptunium, Plutonium, Americium, Curium, Berkelium, Californium, Einsteinium, Fermium, Mendelevium, or Nobelium. In some embodiments, the radioisotope is actinium-225.

In some embodiments, the radioisotope is of a Group 13, Group 14, Group 15, Group 16, or Group 17 metal. In some embodiments, the metal or radioisotope is an isotope of any of Tin, Lead, or Bismuth. In some embodiments, the radioisotope is lead-212.

Various isotopes can be used in a composition or method of the invention. Table 1 lists illustrative isotopes that can be used in a composition and methods of the invention.

TABLE 1

| Isotopes | Estimated Half Life | |
|---|---|---|
| Actinium-225 | 10 | days |
| Bismuth-213 | 46 | min |
| Chromium-51 | 28 | days |
| Cobalt-57 | 272 | days |
| Cobalt-60 | 5.27 | years |
| Copper-64 | 13 | hours |
| Gallium-67 | 78 | hours |
| Iridium-192 | 74 | days |
| Iron-59 | 46 | days |
| Lead-212 | 10.4 | hours |
| Lutetium-177 | 6.7 | days |
| Palladium-103 | 17 | days |
| Samarium-153 | 47 | hours |
| Strontium-89 | 50 | days |
| Strontium-92 | 25 | days |
| Thallium-201 | 73 | hours |
| Yttrium-90 | 64 | hours |

The invention also provides a composition for treating cancer comprising a radioisotope-binding peptide; a radioisotope bound to the radioisotope-binding peptide; and a pharmaceutically-acceptable excipient, wherein the radioisotope decays into a daughter nuclide, and wherein the radioisotope-binding peptide binds the radioisotope with a radioisotope affinity constant, and wherein the radioisotope-binding peptide binds the daughter nuclide with a daughter nuclide affinity constant. In some embodiments, the radioisotope affinity constant is at least 2 times greater than the daughter nuclide affinity constant. In some embodiments, the radioisotope affinity constant is at least 10 times greater than the daughter nuclide affinity constant. In some embodiments, the radioisotope decays into a daughter nuclide with a decay constant of greater than 60 minutes.

Also described herein is a method of treating cancer with a metal or radioisotope comprising administering a composition according to any of the above to a subject in need or want thereof. The cancer can be metastatic. In some embodiments, the cancer is a hematological neoplasm, including leukemias and lymphomas. In some embodiments, the cancer is a solid tumor.

A peptide of the invention can be administered to a subject. Various animals can be subjects of the invention. An animal can be, for example, a mammal, a primate, a vertebrate, a human, dog, a cat, a horse, a cow, a pig, a mouse, a rat, a rabbit, a guinea pig, or a monkey. In some embodiments, a subject is a human. A subject can be of any age, including, for example, elderly adults, adults, adolescents, pre-adolescents, children, toddlers, and infants. A peptide of the invention can be administered to a subject in need or want of cancer treatment.

### Metal Abstraction Peptide (MAP) Tag.

The present disclosure generally relates to metal-binding peptides and methods of using such peptides. These peptides have the ability to bind to metals, which makes them useful for a variety of applications. In particular, the metal-binding peptides have applications in site-specific modulation of peptides or proteins to which the metal-binding peptides are linked. End uses of such modulated peptides or proteins include, for example, imaging, research, therapeutics, pharmaceuticals, chemotherapy, chelation therapy, metal sequestering, and radiotherapy. In particular, end uses can include chemotherapy and radiotherapy for treatment of cancer.

The present disclosure provides a tripeptide having the sequence XC₁C₂, wherein X is any natural or non-natural amino acid or amino acid analogue such that XC₁C₂ is capable of binding a metal in a square planar orientation or square pyramidal orientation or both, and wherein C₁ and C₂ are the same or different, and wherein C₁ and C₂ are each individually chosen from a cysteine and a cysteine-like non-natural amino acid or amino acid analogue. In some embodiments, the metal-binding peptide comprises a sequence XC₁C₂, wherein X is any natural or non-natural amino acid or amino acid analogue, and further wherein C₁ and C₂ are each individually chosen from a cysteine and a sulfur-containing alpha or beta amino acid or amino acid analogue.

The present disclosure also provides a composition comprising a tripeptide having the sequence XC₁C₂ and a metal, wherein the metal is complexed with the tripeptide, wherein X is any natural or non-natural amino acid or amino acid analogue such that XC₁C₂ and the bound metal are in a square planar orientation or square pyramidal orientation or both, wherein C₁ and C₂ are the same or different, and wherein C₁ and C₂ individually are chosen from a cysteine and a cysteine-like non-natural amino acid or amino acid analogue. In some embodiments, the metal-binding peptide comprises a sequence XC₁C₂, wherein X is any natural or non-natural amino acid or amino acid analogue, and further wherein C₁ and C₂ are each individually chosen from a cysteine and a sulfur-containing alpha or beta amino acid or amino acid analogue.

The present disclosure provides methods comprising complexing with a metal any peptide described herein.

In some embodiments, the present disclosure provides peptide motifs that strongly bind with a select metal, referred to as MAP tag(s). In some embodiments, the MAP tag can be attached to another molecule. The MAP tags can be 3 or more amino acid residues, and can be included in longer polypeptides and proteins at the N-terminus, C-terminus, or any position in between. In some embodiments, the MAP tag can be present in a polypeptide or protein configuration that presents the MAP tag for binding with a metal, such as being present in an external loop. The MAP tag can be attached to a non-peptide entity. Non-peptide entities can include carbohydrates and/or covalent linkers. For example, the MAP tag can be attached to a non-peptide entity like a carbohydrate. For example, the carbohydrate can be a component in a glycoprotein. Alternatively, the carbohydrate can be hyaluronic acid or chondroitin. The attachment can be covalent, and can be affected through a linker. In some embodiments, a linker can be a polyethylene glycol group.

A MAP tag can be included within or linked to an antibody or non-antibody protein. In some embodiments, the MAP tag is included with an epidermal growth factor (EGF) protein. In various cancers, EGF receptor (EGFR) is overexpressed. In some embodiments, the metal-peptide complex or radioisotope-metal complex can be incorporated within or linked to an EGF protein. The EGF protein can be targeted for delivery to cancer cells overexpressing the EGF receptor, such as head and neck cancers. In some embodiments, the MAP tag is included within or linked to a modified version of an antibody such as trastuzumab (Herceptin®) for targeted delivery to HER2+ breast cancer cells.

A MAP tag of the present disclosure can be encoded within a gene or nucleotide sequence that provides for targeted delivery of the MAP tag, either before MAP tag complexation with a metal or after complexation with a metal. Targeting can be accomplished using genes, peptides, or other motifs known to be useful for targeting. For example, MAP tags can be incorporated with antibodies, growth factors, and peptides. Additionally, a MAP tag of the invention can be incorporated into a peptide or protein using any synthetic or biosynthetic method for peptide or protein production. In some embodiments, the MAP tag spontaneously reacts with a metal to form a peptide-metal complex. Such peptide-metal complexes can form in solution or via transmetallation or any other process.

Nucleic acids encoding polypeptides or polypeptide fusion proteins/chimeric proteins described herein can be used to construct recombinant expression vectors capable of expressing the polypeptides or polypeptide fusion proteins/chimeric proteins of the present invention. In some embodiments, nucleic acid constructs capable of expressing the protein constructs described herein comprise nucleotide sequences containing transcriptional and translational regulatory information and such sequences are operably linked to nucleotide coding sequences.

Selection of the appropriate vector can depend on: 1) whether the vector is to be used for nucleic acid amplification and/or for nucleic acid expression; 2) the size of the nucleic acid to be inserted into the vector; and 3) the host cell to be transformed with the vector. A vector can contain various components specific to the function thereof (e.g. amplification of nucleic acid or expression of nucleic acid) and the host cell into for which the vector is introduced.

A host cell can be adapted to express one or more peptides or peptide fusion proteins/chimeric proteins described herein. The host cells encompass cells in prokaryotic, eukaryotic, and insect cells. In some embodiments, host cells are capable of modulating the expression of the inserted sequences, or modifying and processing the gene or protein product in the specific fashion desired. For example, expression from certain promoters can be elevated in the presence of certain inducers (e.g., zinc and cadmium ions for metallothionine promoters). In some embodiments, modifications (e.g., phosphorylation) and processing (e.g., cleavage) of peptide products are important for the function of the peptide. Host cells can have characteristic and specific mechanisms for the post-translational processing and modification of a peptide. In some embodiments, host cells secrete minimal amounts of proteolytic enzymes. In some embodiments, host systems of viral origin are utilized to perform the processes described for the host cell.

Various expression vector/host systems can be utilized for the recombinant expression of polypeptides or polypeptide fusion proteins/chimeric proteins described herein. Non-limiting examples of such systems include microorganisms such as bacteria transformed with recombinant bacteriophage DNA, plasmid DNA or cosmid DNA expression vectors containing a nucleic acid sequence encoding peptides or peptide fusion proteins/chimeric proteins described herein, yeast transformed with recombinant yeast expression vectors containing the aforementioned nucleic acid sequence, insect cell systems infected with recombinant virus expression vectors (e.g., baculovirus) containing the aforementioned nucleic acid sequence, plant cell systems infected with recombinant virus expression vectors (e.g., cauliflower mosaic virus (CaMV), tobacco mosaic virus (TMV) or transformed with recombinant plasmid expression vectors (e.g., Ti plasmid) containing the aforementioned nucleic acid sequence, or animal cell systems infected with recombinant virus expression vectors (e.g. adenovirus, vaccinia virus) including cell lines engineered to contain multiple copies of the aforementioned nucleic acid sequence, either stably amplified (e.g., CHO/dhfr, CHO/glutamine synthetase) or unstably amplified in double-minute chromosomes (e.g., murine cell lines).

In the case of cell- or viral-based samples, organisms can be treated prior to purification to preserve and/or release a target polypeptide. In some embodiments, the cells are fixed using a fixing agent. In some embodiments, the cells are lysed. The cellular material can be treated in a manner that does not disrupt a significant proportion of cells, but which removes proteins from the surface of the cellular material, and/or from the interstices between cells. For example, cellular material can be soaked in a liquid buffer, or, in the case of plant material, can be subjected to a vacuum, in order to remove proteins located in the intercellular spaces and/or in the plant cell wall. If the cellular material is a microorganism, proteins can be extracted from the microorganism culture medium. Alternatively, the peptides can be packed in inclusion bodies. The inclusion bodies can further be separated from the cellular components in the medium. In some embodiments, the cells are not disrupted. A cellular or viral peptide that is presented by a cell or virus can be used for the attachment and/or purification of intact cells or viral particles. Peptides can also be synthesized in a cell-free system prior to extraction.

More than one MAP tag can be present in a particular molecule. In some embodiments, the MAP-tag is a tripeptide capable of complexation with metal ions, as described in U.S. Patent Publication 2010/0221839.

### Chemical Structure/Peptide Sequence.

The MAP tags of the present disclosure generally comprise at least three amino acid residues, and are capable of binding a metal. The MAP tags can have a sequence represented by XC₁C₂, in which C₁ and C₂ can be the same or different and can be a cysteine, or a cysteine-like non-natural amino acid, or a cysteine-like amino acid analogue. For example, C₁ and/or C₂ can be a sulfur containing alpha- or beta-amino acid.

In some embodiments, the MAP tag can comprise one of the following sequences: NC₁C₂; Z¹—NC₁C₂—Z²; Z¹—NC₁C₂; NC₁C₂—Z²; QC₁C₂; Z¹-QC₁C₂—Z²; Z¹-QC₁C₂; QC₁C₂—Z²; HC₁C₂; Z¹—HC₁C₂—Z²; Z¹—HC₁C₂; HC₁C₂—Z²; KC₁C₂; Z¹—KC₁C₂—Z²; Z¹—KC₁C₂; KC₁C₂—Z²; RC₁C₂; Z¹—RC₁C₂—Z²; Z¹—RC₁C₂; or RC₁C₂—Z². Any of Z¹, Z², Z³, and Z⁴ can be the same or different. In some embodiments, the MAP tag can comprise one of the following sequences: Z¹—XC₁C₂, XC₁C₂—Z², Z¹—XC₁C₂—Z², XZ³C₁C₂, XC₁Z⁴C₂, XZ³C₁Z⁴C₂, and Z¹—XZ³C₁Z⁴C₂—Z², wherein X is any natural or non-natural amino acid or amino acid analogue, and further wherein C₁ and C₂ are each individually chosen from a cysteine and a sulfur-containing alpha or beta amino acid, and wherein Z¹, Z², Z³, and Z⁴ are each individually a sequence of 1-5 residues, or absent, wherein each residue is independently a natural or non-natural amino acid or analogue thereof. In some embodiments, at least one of Z¹, Z², Z³, and Z⁴ includes a basic amino acid adjacent to X, C₁ or C₂.

Any of Z¹, Z², Z³, and Z⁴ can be 0, 1, 2, 3, 4, 5, 6, 7, 8, 9, or 10 residues, wherein each residues is independently a natural or non-natural amino acid residue or analogue thereof. In some embodiments, Z¹, Z², Z³, and Z⁴ are each individually a sequence of any natural or non-natural amino acid or amino acid analogue. In some embodiments, the sequence of Z¹ is a sequence of 0-1, 0-2, 0-3, 0-4, 0-5, 1-2, 1-3, 1-4, 1-5, 1-6, 1-7, 1-8, 1-9, or 1-10 natural or non-natural amino acid or amino acid analogue long. In some embodiments, the sequence of Z² is a sequence of 0-1, 0-2, 0-3, 0-4, 0-5, 1-2, 1-3, 1-4, 1-5, 1-6, 1-7, 1-8, 1-9, or 1-10 residues long. In some embodiments, the sequence of Z³ is a sequence of 0-1, 0-2, 0-3, 0-4, 0-5, 1-2, 1-3, 1-4, 1-5, 1-6, 1-7, 1-8, 1-9, or 1-10 residues long. In some embodiments, the sequence of Z⁴ is a sequence of 0-1, 0-2, 0-3, 0-4, 0-5, 1-2, 1-3, 1-4, 1-5, 1-6, 1-7, 1-8, 1-9, or 1-10 residues long. Any residue can independently be a natural or non-natural amino acid residue or analogue thereof.

In some embodiments, the invention provides a metal-binding peptide or a radioisotope-binding peptide comprising a sequence XC₁C₂, wherein X is any natural or non-natural amino acid or amino acid analogue, and further wherein C₁ and C₂ are each individually chosen from a cysteine and a sulfur-containing alpha or beta amino acid. In some embodiments, the sequence XC₁C₂ is included in a sequence Z¹—XC₁C₂, wherein Z¹ is any natural or non-natural amino acid or sequence of natural or non-natural amino acids. In some embodiments, the sequence XC₁C₂ is included in a sequence XC₁C₂—Z², wherein Z² is any natural or non-natural amino acid or sequence of natural or non-natural amino acids or analogue thereof. In some embodiments, the sequence XC₁C₂ is included in a sequence Z¹—XC₁C₂—Z², wherein Z¹ and Z² are each individually any natural or non-natural amino acid or sequence of natural or non-natural amino acids. In some embodiments, at least one of Z¹ and Z² includes a basic amino acid adjacent to either X or C₂.

The invention provides a metal-binding peptide or a radioisotope-binding peptide comprising a sequence XZ³C₁C₂, wherein X is any natural or non-natural amino acid or amino acid analogue, and wherein C₁ and C₂ are each individually chosen from a cysteine and a sulfur-containing alpha or beta amino acid, and wherein Z³ is a sequence of 1-5 residues, or absent, wherein each residue is independently a natural or non-natural amino acid or analogue thereof. In some embodiments, the peptide comprises a sequence XC₁Z⁴C₂, wherein X is any natural or non-natural amino acid or amino acid analogue, and wherein C₁ and C₂ are each individually chosen from a cysteine and a sulfur-containing alpha or beta amino acid, and wherein Z⁴ is a sequence of 1-5 residues, or absent, wherein each residue is independently a natural or non-natural amino acid or analogue thereof. In some embodiments, the metal-binding peptide comprises a sequence XZ³C₁Z⁴C₂, wherein X is any natural or non-natural amino acid or amino acid analogue, and wherein C₁ and C₂ are each individually chosen from a cysteine and a sulfur-containing alpha or beta amino acid, and wherein Z³ and Z⁴ are each individually a sequence of 1-5 residues, or absent, wherein each residue is independently a natural or non-natural amino acid or analogues thereof. In some embodiments, at least one of Z³ and Z⁴ is present.

The invention provides for a metal-binding or a radioisotope-binding peptide comprising a sequence Z¹—XZ³C₁Z⁴C₂—Z² wherein X is any natural or non-natural amino acid or amino acid analogue, and wherein C₁ and C₂ are each individually chosen from a cysteine and a sulfur-containing alpha or beta amino acid, wherein Z¹ and Z² are each individually absent or present and are each individually any natural or non-natural amino acid or sequence of natural or non-natural amino acids, and wherein Z³ and Z⁴ are each individually a sequence of 1-5 residues, or absent, wherein each residue is independently a natural or non-natural amino acid or analogues thereof.

In some embodiments, at least one of Z¹, Z², Z³, and Z⁴ above includes a basic amino acid adjacent to X, C₁, or C₂.

### Peptide Lengths.

The peptides of the invention can be incorporated into peptides of various sizes. The peptides of the invention can be about 3 amino acids, about 4 amino acids, about 5 amino acids, about 6 amino acids, about 7 amino acids, about 8 amino acids, about 9 amino acids, about 10 amino acids, about 11 amino acids, about 12 amino acids, about 13 amino acids, about 14 amino acids, about 15 amino acids, about 16 amino acids, about 17 amino acids, about 18 amino acids, about 19 amino acids, about 20 amino acids, no more than 20 amino acids, no more than 25 amino acids, no more than 30 amino acids, no more than 35 amino acids, no more than 40 amino acids, no more than 45 amino acids, no more than 50 amino acids, no more than 55 amino acids, no more than 60 amino acids, no more than 65 amino acids, no more than 70 amino acids, no more than 75 amino acids, no more than 80 amino acids, no more than 85 amino acids, no more than 90 amino acids, no more than 95 amino acids, no more than 100 amino acids, no more than 110 amino acids, no more than 120 amino acids, no more than 130 amino acids, no more than 140 amino acids, no more than 150 amino acids, no more than 160 amino acids, no more than 170 amino acids, no more than 180 amino acids, no more than 190 amino acids, no more than 200 amino acids, no more than 225 amino acids, no more than 250 amino acids, no more than 275 amino acids, no more than 300 amino acids, no more than 325 amino acids, no more than 350 amino acids, no more than 375 amino acids, no more than 400 amino acids, no more than 425 amino acids, no more than 450 amino acids, no more than 475 amino acids, or no more than 500 amino acids.

In some embodiments, the metal-binding peptide or radioisotope-binding peptide comprises at least 20 amino acids. In some embodiments, the metal-binding or radioisotope-binding peptides can adopt a secondary, a tertiary, and or a quaternary structure. In addition, the metal-binding peptide can adopt a tertiary structure under physiological conditions wherein a basic amino acid located some number of amino acids away from C₁ by amino acid sequence is located within some distance (Å) in space from C₁. In some embodiments, the basic amino acid is located within 1 Å, 2 Å, 3 Å, 4 Å, 5 Å, 6 Å, 7 Å, 8 Å, 9 Å, 10 Å, 11 Å, 12 Å, 13 Å, 14 Å, 15 Å, 16 Å, 17 Å, 18 Å, 19 Å, 20 Å, 21 Å, 22 Å, 23 Å, 24 Å, 25 Å, 26 Å, 27 Å, 28 Å, 29 Å, 30 Å, 31 Å, 32 Å, 33 Å, 34 Å, 35 Å, 36 Å, 37 Å, 38 Å, 39 Å, 40 Å, 41 Å, 42 Å, 43 Å, 44 Å, 45 Å, 46 Å, 47 Å, 48 Å, 49 Å, or 50 Å from C₁. In some embodiments, the basic amino acid is located at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 55, at least 60, at least 65, at least 70, at least 75, at least 80, at least 85, at least 90, at least 95, or at least 100 amino acids away from C₁.

The secondary structure of the metal-binding or radioisotope binding peptide can comprise alpha-helices, beta-helices, pi-helices, 3,10 helices, beta-sheets, beta-strands, beta-barrels, or any combination thereof. In some embodiments, a peptide of the invention can be covalently incorporated into a molecule such that the structure of the original beta-sheet and alpha-helices of the molecule are not disrupted. The tertiary structure of a metal-binding or radioisotope binding peptide can be arranged in a multi-subunit complex, or quaternary structure. In some embodiments, the tertiary structure of a metal-binding or radioisotope-binding peptide brings a basic amino acid located at least 17 amino acids away from C₁ within 20 angstroms in space from C₁. In some embodiments, the basic amino acid is selected from the group consisting of arginine and lysine.

### Metal Binding.

Metal binding by MAP tags can be accomplished using atoms in very close proximity. Various conditions can release the metal. Thermal and chemical denaturation of the MAP tag and the material to which the tag is covalently linked permits slow release of the metal. For example, use of extreme conditions (e.g. boiling temperature, denaturants, chelators) can lead to slow release of the metal over a period of time (e.g., several minutes to many hours). In some embodiments, a bound metal is released upon a shift in pH. In some embodiments, a bound radioisotope undergoes radioactive decay, and the daughter nuclide resulting from the radioactive decay is released.

In some embodiments, the MAP tags of the present disclosure, alone or when incorporated into a polypeptide or protein, can complex with a metal to form a MAP tag-metal complex having a square planar/pyramidal geometry. The metal can complex with the MAP tag through 2N:2S coordination.

Non-limiting examples of suitable metals and radioisotopes include Group 3 metals, Group 4 metals, Group 5 metals, Group 6 metals, Group 7 metals, Group 8 metals, Group 9 metals, Group 10 metals, Group 11 metals, Group 12 metals, Group 13 metals, Group 14 metals, Group 15 metals, transition metals, main group metals, the lanthanide metals, the actinide metals, and the transuranic metals. For example, the metal or radioisotope can be selected from Group 10 metals, Group 11 metals, and Group 13 metals. In some embodiments, the metal is a lanthanide metal, an actinide metal, or a transuranic metal. Non-limiting examples of metal include Scandium, Titanium, Vanadium, Chromium, Manganese, Iron, Cobalt, Nickel, Copper, Zinc, Yttrium, Zirconium, Niobium, Molybdenum, Technetium, Ruthenium, Rhodium, Palladium, Silver, Cadmium, Lutetium, Hafnium, Tantalum, Tungsten, Rhenium, Osmium, Iridium, Platinum, Gold, Mercury, Lawrencium, Rutherfordium, Dubnium, Seaborgium, Bohrium, Hassium, Meitnerium, Darmstadtium, Roentgenium, Ununbiumm, Lanthanum, Cerium, Praseodymium, Neodymium, Promethium, Samarium, Europium, Gadolinium, Terbium, Dysprosium, Holmium, Erbium, Thulium, Ytterbium, Lutetium, Actinium, Thorium, Protactinium, Uranium, Neptunium, Plutonium, Americium, Curium, Berkelium, Californium, Einsteinium, Fermium, Mendelevium, Nobelium, Aluminum, Gallium, Indium, Thallium, Ununtrium, Germanium, Tin, Lead, Flerovium, Arsenic, Bismuth, Ununpentium, Polonium, and Livermorium. In some embodiments, the metal is platinum.

Non-limiting examples of radioisotopes include alpha emitters, beta emitters, positron emitters, and gamma emitters. In some embodiments, the metal or radioisotope is selected from the group consisting of actinium, americium, bismuth, cadmium, cesium, cobalt, europium, gadolinium, iridium, lead, lutetium, manganese, palladium, polonium, radium, ruthenium, samarium, strontium, technetium, thallium, and yttrium. In some embodiments, the metal is actinium, bismuth, lead, radium, strontium, samarium, or yttrium. In some embodiments, the radioisotope is actinium-225 or lead-212.

In some embodiments, a MAP tag is capable of binding metals with high affinity. A MAP tag can also be capable of abstracting a metal from various compositions ranging from fluids to solids. Consequently, the ability of MAP tags to abstract the metal, rather than share coordination, make the tags amenable for use in keeping a specific metal separate from the in vivo environment until release. In some embodiments, initial binding is best accomplished using a partial chelator as opposed to a chelator that coordinates at all available binding sites on the metal. In some embodiments, chelators like EDTA that coordinate a metal at all available binding sites on the metal proceed much more slowly. Non-limiting examples of suitable partial chelators include nitrilotriacetic acid (NTA), iminodiacetic acid (IDA), sulfate, histidine, and imidazole.

In some embodiments, the release rate of a MAP sequence can be tailored by controlling the pH. For example, higher pH (e.g., above 7) can provide stable metal-peptide complexes. In some embodiments, the metal peptide complex is stable at pH 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9 or above. In some embodiments, the metal peptide complex is unstable at pH below 7, such as 6.9, 6.8, 6.7, 6.6, 6.5, 6.4, 6.3, 6.2, 6.1, 6, 5.9, 5.8, 5.7, 5.6, 5.5, 5.4, 5.3, 5.2, 5.1, 5, 4.9, 4.8, 4.7, 4.6, 4.5, 4.4, 4.3, 4.2, 4.1, 4, or below. The stability also can depend on and be controlled by the particular amino acid chosen for the X or Z¹-Z⁴ positions, for example, as a function of pH.

### Chemotherapy and Radiotherapy.

Also provided herein is a method of treating cancer with a metal or radioisotope comprising administering a composition according to any of the above to a subject in need or want thereof. The cancer can be metastatic. In some embodiments, the cancer is a hematological neoplasm, including leukemias and lymphomas. In some embodiments, the cancer is a solid tumor.

In some embodiments the invention provides a metal-binding peptide composition for use in treating cancer in a subject in need or want of relief thereof, wherein the metal-binding peptide for use in treating a cancer comprises a sequence Z¹—XZ³C₁Z⁴/C₂—Z², wherein X is any natural or non-natural amino acid or amino acid analogue, and wherein C₁ and C₂ are each individually chosen from a cysteine and a sulfur-containing alpha or beta amino acid, and wherein Z¹, Z², Z³, and Z⁴ are each individually a sequence of 1-5 residues, or absent, wherein each residue is independently a natural or non-natural amino acid or analogue thereof, and wherein the metal-binding peptide composition is bound to a metal. In some embodiments the metal-binding peptide for treating cancer comprises a pharmaceutically-acceptable excipient. In some embodiments, the composition comprises a unit dosage form.

In some embodiments, the invention is a use of a metal-binding peptide in the preparation of a medicament for treating a cancer in a subject in need or want of relief thereof, wherein the metal-binding peptide for use in treating a cancer comprises a sequence Z¹—XZ³C₁Z⁴/C₂—Z², wherein X is any natural or non-natural amino acid or amino acid analogue, and wherein C₁ and C₂ are each individually chosen from a cysteine and a sulfur-containing alpha or beta amino acid, and wherein Z¹, Z², Z³, and Z⁴ are each individually a sequence of 1-5 residues, or absent, wherein each residue is independently a natural or non-natural amino acid or analogue thereof, and wherein the metal-binding peptide is bound to a metal. In some embodiments the medicament comprises a pharmaceutically-acceptable excipient. In some embodiments, the use a medicament comprises a unit dosage form.

The present invention provides for compositions for use in treating cancer. Such compositions comprise a metal-binding peptide and a metal bound to the metal-binding peptide. In some embodiments, the metal is a radioisotope. The compositions can further comprise a pharmaceutically-acceptable excipient. In some embodiments, the compositions further comprise buffer. In some embodiments, the buffer is a phosphate buffer. In some embodiments, the composition has a pH between about 7 and about 8. In some embodiments, the composition has a pH of about 7.4.

In some embodiments, the composition for treating cancer comprises a metal-binding peptide; a metal bound to the metal-binding peptide; and a pharmaceutically-acceptable excipient, wherein the metal-binding peptide binds the metal with an affinity constant, wherein the affinity constant is at least 2 times greater at a pH above 7 than an affinity constant at a pH below 6. In various embodiments, the affinity constant at higher pH is at least 10 times greater than the affinity constant at lower pH. In some embodiments, the affinity constant at a pH between 7 and 8 is at least 10 times greater than an affinity constant at a pH between 4 and 6.

In some embodiments, the composition for treating cancer comprises a metal-binding peptide; a metal bound to the metal-binding peptide; and a pharmaceutically-acceptable excipient; wherein the composition contains a concentration of metal-binding peptide and a concentration of metal, and wherein the metal-binding peptide binds the metal to yield a concentration of peptide with bound metal and a concentration of peptide without bound metal and a ratio of the concentrations, wherein the ratio is at least two times greater at a pH above 7 than the ratio at a pH below 6 for a constant concentration of metal-binding peptide and metal. In some embodiments, the ratio is at least five times greater at a pH above 7 than the ratio at a pH below 6. In some embodiments, the concentration of metal-binding peptide and the concentration of metal are in a ratio of about 1:about 1 or greater than 1:about 1. In some embodiments, the concentration of metal-binding peptide and the concentration of metal are in a ratio ranging from about 1:about 1 to about 2:about 1. In some embodiments, the ratio is at least five times greater at a pH between 7 and 8 than the ratio at a pH between 4 and 6.

In some embodiments, the invention provides for a composition for treating cancer comprising a radioisotope-binding peptide; a radioisotope bound to the radioisotope-binding peptide; and a pharmaceutically-acceptable excipient, wherein the radioisotope decays into a daughter nuclide, and wherein the radioisotope-binding peptide binds the radioisotope with a radioisotope affinity constant, and wherein the radioisotope-binding peptide binds the daughter nuclide with a daughter nuclide affinity constant. In some embodiments, the radioisotope affinity constant is at least 2 times greater than the daughter nuclide affinity constant. In some embodiments, the radioisotope affinity constant is at least 10 times greater than the daughter nuclide affinity constant. In some embodiments, the radioisotope decays into a daughter nuclide with a decay constant of greater than 60 minutes. As the binding constant decreases upon radioactive decay of the radioisotope, the daughter nuclide is effectively released from the radioisotope-peptide complex. In some embodiments, the peptide is used for selective delivery of the radioisotope to the vicinity of a cancer cell, or within a cancer cell.

In some embodiments, the invention provides the metal-binding peptide with bound metal or the radioisotope-binding peptide with bound radioisotope to achieve a local concentration near a solid tumor or within cancer cells and an average serum concentration within four hours of administration, wherein the local concentration near the solid tumor is at least twice the average serum concentration. Upon being located near or in cancer cells, the metal or radioisotope is released by a change in pH near the tumor or within tumor cells. For example, the pH of blood is generally considered to be about 7.4, and can vary according to location, such as in veins or arteries. Similarly, the pH of the nucleus is about 7.4. In contrast, the pH of a late endosome is about 5.5, and the pH of a lysosome is about 4.8. The pH of the gastrointestinal tract varies considerably (saliva pH 6.0-7.4; gastric juice pH 1.5-3.5; small intestine pH 6.0-6.5; colon pH 5.5-7). Thus, when the peptide-metal complex has a pH-dependent affinity constant, the variation of pH in various compartments in vivo can provide for targeted delivery and release of the bound metal. In some embodiments, the metal-peptide complex or radioisotope-peptide complex is taken up into a cell through endocytotic uptake into a cancer cell.

In some embodiments, the targeted delivery of metal-peptide complexes or radioisotope-peptide complexes allows for reduction in side effects of cancer treatment for a constant amount of therapeutic agent, or delivery of increased amount of therapeutic agent for greater therapeutic efficacy, or both.

In some embodiments, the compositions and methods of the invention are used as an adjuvant therapy. In some embodiments, the compositions and methods are used as an adjuvant therapy in the treatment of cancer.

A MAP tag of the invention can be used to specifically deliver a radioisotope to a cancerous cell. A MAP tag of the invention can be targeted to a tumor associated antigen. Non-limiting examples of cancer biomarkers that can be targeted by compositions and methods of the invention are described in Table 2.

TABLE 2

| Biomarker | Type | Source | Cancer Type |
|---|---|---|---|
| α-Fetoprotein | Glycoprotein | Serum | Nonseminomatous |
| | | | testicular |
| Human chorionic | Glycoprotein | Serum | Testicular |
| gonadotropin-β | | | |
| CA19-9 | Carbohydrate | Serum | Pancreatic |
| CA125 | Glycoprotein | Serum | Ovarian |
| CEA | Protein | Serum | Colon |
| Epidermal growth | Protein | Colon | Colon |
| factor receptor (EGFR) | | | |
| PSA | Protein | Serum | Prostate |
| CA15-3 | Glycoprotein | Serum | Breast |
| CA27-29 | Glycoprotein | Serum | Breast |
| HER2/NEU | Protein | Serum | Breast |
| Folate receptor alpha | Protein | Ovarian | Ovarian |
| (Frα) | | | |

Non-limiting examples of cancers that can be treated with a MAP tag of the invention can include: acute lymphoblastic leukemia, acute myeloid leukemia, adrenocortical carcinoma, AIDS-related cancers, AIDS-related lymphoma, anal cancer, appendix cancer, astrocytomas, basal cell carcinoma, bile duct cancer, bladder cancer, bone cancers, brain tumors, such as cerebellar astrocytoma, cerebral astrocytoma/malignant glioma, ependymoma, medulloblastoma, supratentorial primitive neuroectodermal tumors, visual pathway and hypothalamic glioma, breast cancer, bronchial adenomas, Burkitt lymphoma, carcinoma of unknown primary origin, central nervous system lymphoma, cerebellar astrocytoma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma, germ cell tumors, gallbladder cancer, gastric cancer, gastrointestinal carcinoid tumor, gastrointestinal stromal tumor, gliomas, hairy cell leukemia, head and neck cancer, heart cancer, hepatocellular (liver) cancer, Hodgkin lymphoma, Hypopharyngeal cancer, intraocular melanoma, islet cell carcinoma, Kaposi sarcoma, kidney cancer, laryngeal cancer, lip and oral cavity cancer, liposarcoma, liver cancer, lung cancers, such as non-small cell and small cell lung cancer, lymphomas, leukemias, macroglobulinemia, malignant fibrous histiocytoma of bone/osteosarcoma, medulloblastoma, melanomas, mesothelioma, metastatic squamous neck cancer with occult primary, mouth cancer, multiple endocrine neoplasia syndrome, myelodysplastic syndromes, myeloid leukemia, nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer, ovarian germ cell tumor, pancreatic cancer, pancreatic cancer islet cell, paranasal sinus and nasal cavity cancer, parathyroid cancer, penile cancer, pharyngeal cancer, pheochromocytoma, pineal astrocytoma, pineal germinoma, pituitary adenoma, pleuropulmonary blastoma, plasma cell neoplasia, primary central nervous system lymphoma, prostate cancer, rectal cancer, renal cell carcinoma, renal pelvis and ureter transitional cell cancer, retinoblastoma, rhabdomyosarcoma, salivary gland cancer, sarcomas, skin cancers, skin carcinoma merkel cell, small intestine cancer, soft tissue sarcoma, squamous cell carcinoma, stomach cancer, T-cell lymphoma, throat cancer, thymoma, thymic carcinoma, thyroid cancer, trophoblastic tumor (gestational), cancers of unknown primary site, urethral cancer, uterine sarcoma, vaginal cancer, vulvar cancer, Waldenström macroglobulinemia, and Wilms tumor.

In some embodiments, the compositions and methods of the invention are linked to an antibody. Non-limiting examples of antibodies that can be linked to a MAP tag of the invention are described in Table 3.

TABLE 3

| | | | Indication |
|---|---|---|---|
| Antibody | Brand name | Target | (Targeted disease) |
| Abciximab | ReoPro TM | inhibition of glycoprotein | Cardiovascular disease |
| | | IIb/IIIa | |
| Adalimumab | Humira TM | inhibition of TNF-α | Several auto-immune disorders |
| | | signaling | |
| Alemtuzumab | Campath TM | CD52 | Chronic lymphocytic leukemia |
| Basiliximab | Simulect TM | IL-2Rα receptor (CD25) | Transplant rejection |
| Belimumab | Benlysta TM | inhibition of B-cell | Systemic lupus erythematosus |
| | | activating factor | |
| Bevacizumab | Avastin TM | Vascular endothelial | Colorectal cancer, Age related |
| | | growth factor (VEGF) | macular degeneration (off- |
| | | | label) |
| Brentuximab | Adcetris TM | CD30 | Anaplastic large cell lymphoma |
| vedotin | | | (ALCL) and Hodgkin |
| | | | lymphoma |
| Canakinumab | Ilaris TM | IL-1β | Cryopyrin-associated periodic |
| | | | syndrome (CAPS) |
| Certolizumab | Cimzia TM | inhibition of TNF-α | Crohn's disease |
| pegol[19] | | signaling | |
| Cetuximab | Erbitux TM | epidermal growth factor | Colorectal cancer, Head and |
| | | receptor (EGFR) | neck cancer |
| Daclizumab | Zenapax TM | IL-2Rα receptor (CD25) | Transplant rejection |
| Denosumab | Prolia, Xgeva | RANK Ligand inhibitor | Postmenopausal osteoporosis, |
| | TM | | Solid tumor's bony metastases |
| Eculizumab | Soliris TM | Complement system | Paroxysmal nocturnal |
| | | protein C5 | hemoglobinuria |
| Efalizumab | Raptiva TM | CD11a | Psoriasis |
| Gemtuzumab | Mylotarg TM | CD33 | Acute myelogenous leukemia |
| | | | (with calicheamicin) |
| Golimumab | Simponi TM | TNF-alpha inhibitor | Rheumatoid arthritis, Psoriatic |
| | | | arthritis, and Ankylosing |
| | | | spondylitis |
| Ibritumomab | Zevalin TM | CD20 | Non-Hodgkin lymphoma (with |
| tiuxetan | | | yttrium-90 or indium-111) |
| Infliximab | Remicade TM | inhibition of TNF-α | Several autoimmune disorders |
| | | signaling | |
| Ipilimumab | Yervoy TM | blocks CTLA-4 | Melanoma |
| (MDX-101) | | | |
| Muromonab-CD3 | Orthoclone TM | T cell CD3 Receptor | Transplant rejection |
| | OKT3 TM | | |
| Natalizumab | Tysabri TM | alpha-4 (α4) integrin, | Multiple sclerosis and Crohn's |
| | | | disease |
| Ofatumumab | Arzerra TM | CD20 | Chronic lymphocytic leukemia |
| Omalizumab | Xolair TM | immunoglobulin E (IgE) | mainly allergy-related asthma |
| Palivizumab | Synagis TM | epitope of the RSV F | Respiratory Syncytial Virus |
| | | protein | |
| Panitumumab | Vectibix TM | epidermal growth factor | Colorectal cancer |
| | | receptor (EGFR) | |
| Ranibizumab | Lucentis TM | Vascular endothelial | Macular degeneration |
| | | growth factor A (VEGF- | |
| | | A) | |
| Rituximab | Rituxan TM | CD20 | Non-Hodgkin lymphoma |
| | Mabthera TM | | |
| Tocilizumab (or | Actemra TM | Anti-IL-6R | Rheumatoid arthritis |
| Atlizumab) | RoActemra TM | | |
| Tositumomab | Bexxar TM | CD20 | Non-Hodgkin lymphoma |
| Trastuzumab | Herceptin TM | ErbB2 (HER2) | Breast cancer |

Therapeutic monoclonal antibodies (mAbs) can be used for treating cancer, immunological disorders, and a plurality of diseases. The major targets of current mAb products include, for example, Epidermal Growth Factor Receptor (EGFR), Human Epidermal Growth Factor receptor 2 (HER2), Vascular Endothelial Growth Factor (VEGF), and CD20. Numerous cancers are EGFR positive (EGFR+) and can be differentially treated according to an EGFR+ diagnosis. Cetuximab and panitumumab products are approved for the treatment of head and neck squamous cell carcinomas and can be used to treat metastatic colon cancer. Trastuzumab is approved by the FDA and Trastuzumab can be used as a primary therapy to treat HER2 positive (HER2+) breast cancers. In some embodiments, the MAP tag of the invention increases the efficacy of the therapies of Table 3 by modulating the colloidal properties of the antibody. In some embodiments a MAP tag of the invention can be added to an antibody to increase the efficacy of the antibody as a therapy.

In some embodiments, the compositions and methods of the invention release a metal bound to the MAP tag in various physiological environments. Non-limiting examples of pH in different environments of living systems are described in Table 4.

TABLE 4

| Physiological environment | pH |
|---|---|
| Gastric acid | 1 |
| Lysosomes | 4.5 |
| Granules of chromaffin cells | 5.5 |
| Human skin | 5.5 |
| Urine | 6 |
| Cytosol | 7.2 |
| Cerebrospinal fluid (CSF) | 7.5 |
| Blood | 7.34-7.45 |
| Mitochondrial matrix | 7.5 |
| Pancreas (secretions) | 8.1 |

In some embodiments, the invention provides a method of providing a metal to a subject in need or want thereof, the method comprising administering to the subject: i) a metal-binding peptide, wherein the metal-binding peptide comprises a sequence Z¹—XZ³C₁Z⁴C₂—Z², wherein X is any natural or non-natural amino acid or amino acid analogue, and wherein C₁ and C₂ are each individually chosen from a cysteine and a sulfur-containing alpha or beta amino acid, and wherein Z¹, Z², Z³, and Z⁴ are each individually a sequence of 1-5 residues, or absent, wherein each residue is independently a natural or non-natural amino acid or analogue thereof; and ii) the metal, wherein the metal is bound to the metal-binding peptide, wherein the metal-binding peptide releases the metal in a physiological environment, wherein the release is associated with a decrease in pH.

The metal can be released from the metal-binding peptide as the pH of the surrounding physiological environment changes. For example, the pH can change as the metal-peptide complex passes from one physiological environment to another. As the pH decreases, the peptide can release the metal partially or quantitatively. The release can be reversible or irreversible at a given pH. The metal can be any metal herein, for example, platinum. In some embodiments, the metal is not a metal described herein, for example, in some embodiments the metal is not nickel.

The release can occur after a decrease in pH of about 0.1, about 0.2, about 0.3, about 0.4, about 0.5, about 0.6, about 0.7, about 0.8, about 0.9, about 1, about 1.1, about 1.2, about 1.3, about 1.4, about 1.5, about 1.6, about 1.7, about 1.8, about 1.9, about 2, about 2.1, about 2.2, about 2.3, about 2.4, about 2.5, about 2.6, about 2.7, about 2.8, about 2.9, about 3, about 3.1, about 3.2, about 3.3, about 3.4, about 3.5, about 3.6, about 3.7, about 3.8, about 3.9, about 4, about 4.1, about 4.2, about 4.3, about 4.4, about 4.5, about 4.6, about 4.7, about 4.8, about 4.9, about 5, about 5.1, about 5.2, about 5.3, about 5.4, about 5.5, about 5.6, about 5.7, about 5.8, about 5.9, about 6, about 6.1, about 6.2, about 6.3, about 6.4, about 6.5, about 6.6, about 6.7, about 6.8, about 6.9, or about 7 pH units.

The release can occur after a decrease in pH of at least 0.1, at least 0.2, at least 0.3, at least 0.4, at least 0.5, at least 0.6, at least 0.7, at least 0.8, at least 0.9, at least 1, at least 1.1, at least 1.2, at least 1.3, at least 1.4, at least 1.5, at least 1.6, at least 1.7, at least 1.8, at least 1.9, at least 2, at least 2.1, at least 2.2, at least 2.3, at least 2.4, at least 2.5, at least 2.6, at least 2.7, at least 2.8, at least 2.9, at least 3, at least 3.1, at least 3.2, at least 3.3, at least 3.4, at least 3.5, at least 3.6, at least 3.7, at least 3.8, at least 3.9, at least 4, at least 4.1, at least 4.2, at least 4.3, at least 4.4, at least 4.5, at least 4.6, at least 4.7, at least 4.8, at least 4.9, at least 5, at least 5.1, at least 5.2, at least 5.3, at least 5.4, at least 5.5, at least 5.6, at least 5.7, at least 5.8, at least 5.9, at least 6, at least 6.1, at least 6.2, at least 6.3, at least 6.4, at least 6.5, at least 6.6, at least 6.7, at least 6.8, at least 6.9, or at least 7 pH units.

### Pharmaceutical Formulations.

As described above, the invention provides compositions that have binding properties useful for the treatment of cancer by controlled delivery and release of metals and radioisotopes.

Pharmaceutical compositions can be administered in therapeutically-effective amounts as pharmaceutical compositions by any form and route known in the art including, for example, intravenous, subcutaneous, intramuscular, oral, rectal, parenteral, ophthalmic, pulmonary, transdermal, vaginal, otic, nasal, and topical administration. Administration by any pharmaceutically acceptable route can comprise chemotherapy or radiotherapy treatments.

A pharmaceutical composition can be administered in a local or systemic manner, for example, via injection of the compound directly into an organ, optionally in a depot or sustained release formulation. Pharmaceutical compositions can be provided in the form of a rapid release formulation, in the form of an extended release formulation, or in the form of an intermediate release formulation. A rapid release form can provide an immediate release. An extended release formulation can provide a controlled release or a sustained delayed release.

For oral administration, pharmaceutical compositions can be formulated by combining the active compounds with pharmaceutically acceptable carriers or excipients. Such carriers can be used to formulate liquids, gels, syrups, elixirs, slurries, suspensions and the like, for oral ingestion by a subject. Non-limiting examples of solvents used in an oral dissolvable formulation can include water, ethanol, isopropanol, saline, physiological saline, DMSO, dimethylformamide, potassium phosphate buffer, phosphate buffer saline (PBS), sodium phosphate buffer, 4-2-hydroxyethyl-1-piperazineethanesulfonic acid buffer (HEPES), 3-(N-morpholino)propanesulfonic acid buffer (MOPS), piperazine-N,N′-bis(2-ethanesulfonic acid) buffer (PIPES), and saline sodium citrate buffer (SSC). Non-limiting examples of co-solvents used in an oral dissolvable formulation can include sucrose, urea, cremaphor, DMSO, and potassium phosphate buffer.

Pharmaceutical preparations can be formulated for intravenous administration. The pharmaceutical compositions can be in a form suitable for parenteral injection as a sterile suspension, solution or emulsion in oily or aqueous vehicles, and can contain formulatory agents such as suspending, stabilizing and/or dispersing agents. Pharmaceutical formulations for parenteral administration include aqueous solutions of the active compounds in water-soluble form. Suspensions of the active compounds can be prepared as oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. The suspension can also contain suitable stabilizers or agents which increase the solubility of the compounds to allow for the preparation of highly concentrated solutions. Alternatively, the active ingredient can be in powder form for constitution with a suitable vehicle, e.g., sterile pyrogen-free water, before use.

The active compounds can be administered topically and can be formulated into a variety of topically administrable compositions, such as solutions, suspensions, lotions, gels, pastes, medicated sticks, balms, creams, and ointments. Such pharmaceutical compositions can contain solubilizers, stabilizers, tonicity enhancing agents, buffers and preservatives.

The compounds can also be formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as a mixture of fatty acid glycerides, optionally in combination with cocoa butter, is first melted.

The pharmaceutical compositions of the present invention can comprise a pharmaceutically-acceptable excipient, which includes, for example, any and all solvents, diluents, or other liquid vehicle, dispersion or suspension aids, surface active agents, isotonic agents, thickening or emulsifying agents, preservatives, solid binders, and lubricants, as suited to the particular dosage form desired. Remington's Pharmaceutical Sciences, Sixteenth Edition, E. W. Martin (Mack Publishing Co., Easton, Pa., 1980) discloses various excipients used in formulating pharmaceutical compositions and known techniques for the preparation thereof. Non-limiting examples of materials that can serve as pharmaceutically acceptable excipients include sugars, such as lactose, glucose, and sucrose; starches, such as corn starch and potato starch; cellulose and derivatives, such as sodium carboxymethyl cellulose, ethyl cellulose, and cellulose acetate; powdered tragacanth; malt; gelatine; talc; lubricants such as cocoa butter, sodium lauryl sulfate, magnesium stearate, and suppository waxes; oils, such as peanut oil, cottonseed oil, safflower oil, sesame oil, olive oil, corn oil, and soybean oil; glycols; such as propylene glycol; esters such as ethyl oleate, and ethyl laurate; agar; buffering agents, such as magnesium hydroxide and aluminum hydroxide; alginic acid; pyrogenfree water; isotonic saline; Ringer's solution; ethyl alcohol; phosphate buffer solutions; coloring agents; releasing agents; coating agents; sweetening, flavoring, and perfuming agents; preservatives; and antioxidants.

Injectable preparations, for example, sterile injectable aqueous or oleaginous suspensions, can be formulated using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Non-limiting examples of acceptable vehicles and solvents that can be employed are water, Ringer's solution, U.S.P., and isotonic sodium chloride solution. Sterile, fixed oils can be employed as a solvent or suspending medium. Any bland fixed oil can be employed, including synthetic mono- or diglycerides. Fatty acids, such as oleic acid, can be are used in the preparation of injectables.

Injectable formulations can be sterilized, for example, by filtration through a bacterial-retaining filter, or by incorporating sterilizing agents in the form of sterile solid compositions, which can be dissolved or dispersed in sterile water or other sterile injectable medium prior to use.

Pharmaceutical compositions of the present invention can be formulated and employed in combination therapies. The pharmaceutical compositions can be formulated with or administered concurrently with, prior to, or subsequently to, one or more other desired therapeutics or medical procedures. The particular combination of therapies (therapeutics or procedures) to employ in a combination regimen can account for compatibility of the desired therapeutics and/or procedures and the desired therapeutic effect to be achieved. The therapies employed can achieve a desired effect for the same disorder (for example, an inventive composition can be administered concurrently with another chemotherapy or radiotherapy agent), or can achieve different effects (e.g., control of any adverse effects).

Kits are provided for carrying out the methods of administering the disclosed compositions to subjects in need thereof. Such kits can include a number of unit dosages, such as a 30 day supply, or a multi-course treatment regimen. Optionally associated with such container(s) can be a notice in the form prescribed by a governmental agency regulating the manufacture, use, or sale of pharmaceutical products, which notice reflects approval by the agency of manufacture, use, or sale for human administration. In some embodiments, a kit includes written instructions on the use of the metal-binding peptide. The written material can be, for example, a label. The written material can suggest methods of administration. The instructions provide the subject and the supervising physician with the best guidance for achieving the optimal clinical outcome from the administration of the therapy. In some embodiments, the composition and methods of the invention are provided in a unit dosage form.

### Dosing of Pharmaceutical Formulations.

Dosing for the metal-MAP complex, in the methods of the invention can vary based on the subject. The dose can range from about 1×10⁻¹⁰ g to about 5000 mg. Dose range can depend on the form of form and/or route of administration. For example, for systemic administration, non-limiting examples of dose ranges are, e.g. about 1 to about 5000 mg, or about 1 to about 3000 mg, or about 1 to about 2000 mg, or about 1 to about 1000 mg, or about 1 to about 500 mg, or about 1 to about 100 mg, or about 10 to about 5000 mg, or about 10 to about 3000 mg, or about 10 to about 2000 mg, or about 10 to about 1000 mg, or about 10 to about 500 mg, or about 10 to about 200 mg, or about 10 to about 100 mg, or about 20 to about 2000 mg, or about 20 to about 1500 mg, or about 20 to about 1000 mg, or about 20 to about 500 mg, or about 20 to about 100 mg, or about 50 to about 5000 mg, or about 50 to about 4000 mg, or about 50 to about 3000 mg, or about 50 to about 2000 mg, or about 50 to about 1000 mg, or about 50 to about 500 mg, or about 50 to about 100 mg, about 100 to about 5000 mg, or about 100 to about 4000 mg, or about 100 to about 3000 mg, or about 100 to about 2000 mg, or about 100 to about 1000 mg, or about 100 to about 500 mg. In some embodiments, the dose is about 0.1 mg, about 1 mg, about 5 mg, about 10 mg, about 25 mg, about 20 mg, about 15 mg, about 30 mg, about 40 mg, about 50 mg, about 60 mg, about 70 mg, about 80 mg, about 90 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg.

### Pharmaceutically Acceptable Salts.

The invention provides the use of pharmaceutically-acceptable salts of any therapeutic compound described herein. Pharmaceutically-acceptable salts include, for example, acid-addition salts and base-addition salts. The acid that is added to the compound to form an acid-addition salt can be an organic acid or an inorganic acid. A base that is added to the compound to form a base-addition salt can be an organic base or an inorganic base. In some embodiments, a pharmaceutically-acceptable salt is a metal salt. In some embodiments, a pharmaceutically-acceptable salt is an ammonium salt.

Metal salts can arise from the addition of an inorganic base to a compound of the invention. The inorganic base consists of a metal cation paired with a basic counterion, such as, for example, hydroxide, carbonate, bicarbonate, or phosphate. The metal can be an alkali metal, alkaline earth metal, transition metal, or main group metal. In some embodiments, the metal is lithium, sodium, potassium, cesium, cerium, magnesium, manganese, iron, calcium, strontium, cobalt, titanium, aluminum, copper, cadmium, or zinc.

In some embodiments, a metal salt is a lithium salt, a sodium salt, a potassium salt, a cesium salt, a cerium salt, a magnesium salt, a manganese salt, a iron salt, a calcium salt, a strontium salt, a cobalt salt, a titanium salt, an aluminum salt, a copper salt, a cadmium salt, or a zinc salt.

Ammonium salts can arise from the addition of ammonia or an organic amine to a compound of the invention. In some embodiments, the organic amine is triethyl amine, diisopropyl amine, ethanol amine, diethanol amine, triethanol amine, morpholine, N-methylmorpholine, piperidine, N-methylpiperidine, N-ethylpiperidine, dibenzylamine, piperazine, pyridine, pyrrazole, pipyrrazole, imidazole, pyrazine, or pipyrazine.

In some embodiments, an ammonium salt is a triethyl amine salt, a diisopropyl amine salt, an ethanol amine salt, a diethanol amine salt, a triethanol amine salt, a morpholine salt, an N-methylmorpholine salt, a piperidine salt, an N-methylpiperidine salt, an N-ethylpiperidine salt, a dibenzylamine salt, a piperazine salt, a pyridine salt, a pyrrazole salt, a pipyrrazole salt, an imidazole salt, a pyrazine salt, or a pipyrazine salt.

Acid addition salts can arise from the addition of an acid to a compound of the invention. In some embodiments, the acid is organic. In some embodiments, the acid is inorganic. In some embodiments, the acid is hydrochloric acid, hydrobromic acid, hydroiodic acid, nitric acid, nitrous acid, sulfuric acid, sulfurous acid, a phosphoric acid, isonicotinic acid, lactic acid, salicylic acid, tartaric acid, ascorbic acid, gentisinic acid, gluconic acid, glucaronic acid, saccaric acid, formic acid, benzoic acid, glutamic acid, pantothenic acid, acetic acid, propionic acid, butyric acid, fumaric acid, succinic acid, methanesulfonic acid, ethanesulfonic acid, benzenesulfonic acid, p-toluenesulfonic acid, citric acid, oxalic acid, or maleic acid.

In some embodiments, the salt is a hydrochloride salt, a hydrobromide salt, a hydroiodide salt, a nitrate salt, a nitrite salt, a sulfate salt, a sulfite salt, a phosphate salt, isonicotinate salt, a lactate salt, a salicylate salt, a tartrate salt, an ascorbate salt, a gentisinate salt, a gluconate salt, a glucaronate salt, a saccarate salt, a formate salt, a benzoate salt, a glutamate salt, a pantothenate salt, an acetate salt, a propionate salt, a butyrate salt, a fumarate salt, a succinate salt, a methanesulfonate (mesylate) salt, an ethanesulfonate salt, a benzenesulfonate salt, a p-toluenesulfonate salt, a citrate salt, an oxalate salt, or a maleate salt.

### EXAMPLES

### Example 1

### Incorporation of Metal into the MAP Tag

In general, platinum is spectroscopically silent. While other metals provide a colored complex easily observed with both the eye and with common spectroscopic techniques, Pt-NCC is less convenient to characterize. For this reason, the complex was first characterized with nickel. Nickel and platinum have similar electronic properties, yet nickel provides a colored complex that allows more simple characterization. The peptides NCC and GGNCC (SEQ ID NO.:1) were used for preliminary studies.

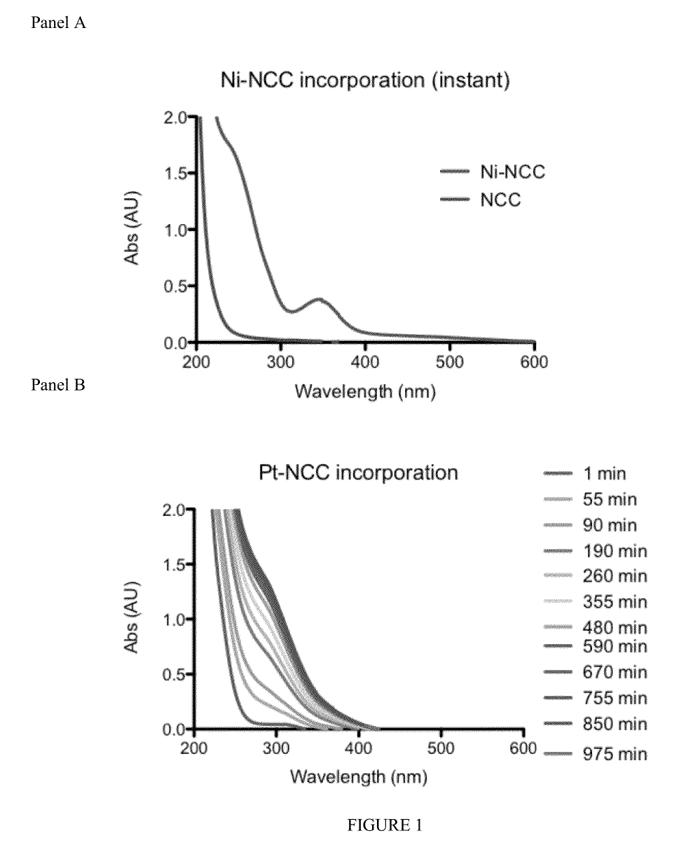

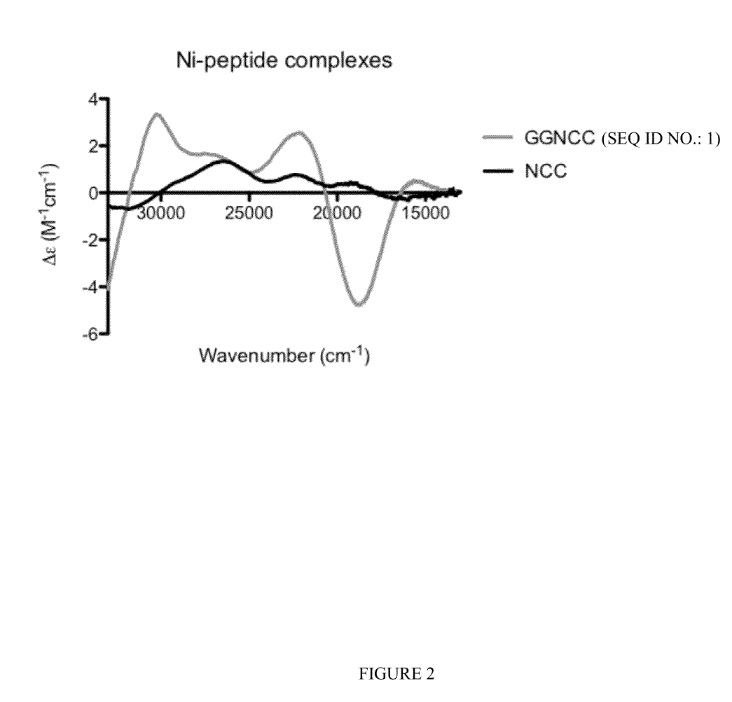

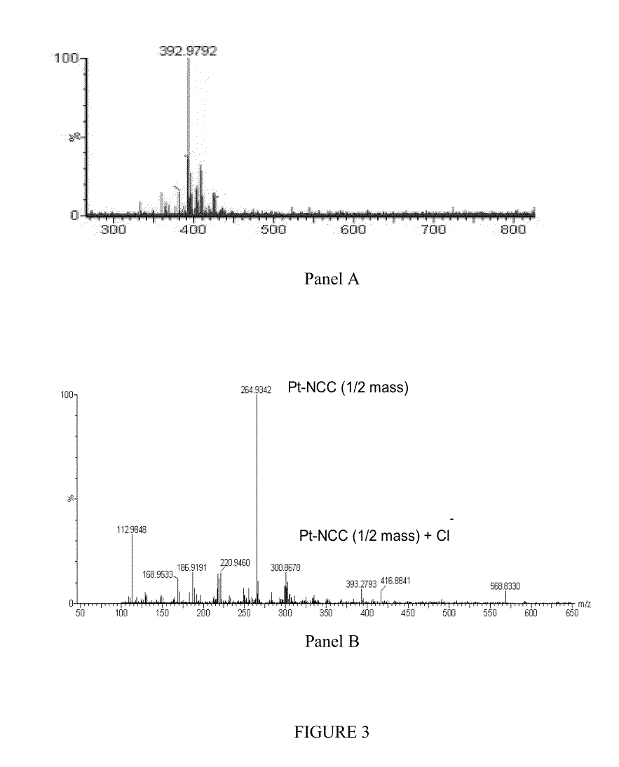

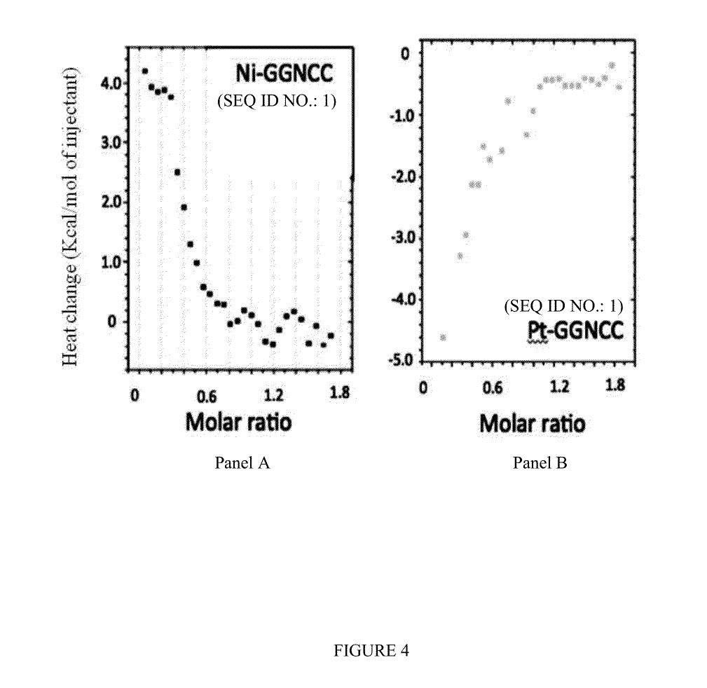

Metal is incorporated into the tag via reaction with a metal ion chelated with nitrilotriacetic acid (NTA). Incorporation of nickel and platinum (FIG. 1, panel A and panel B respectively) was monitored and/or quantified with absorption spectroscopy (FIG. 1). The formation of Ni-peptide complexes was monitored and/or quantified with circular dichroism (FIG. 2). Metal abstraction of nickel and platinum by a NCC peptide was demonstrated by mass spectrometry (FIG. 3, panel A and panel B respectively). The thermodynamic parameters of interaction of a Ni-GGNCC (SEQ ID NO.:1) (FIG. 4, panel A) and a Pt-GGNCC (SEQ ID NO.:1) (FIG. 4, panel B) were determined by isothermal titration calorimetry, and/or atomic absorption spectroscopy. These methods were also used to measure the stability of the complex under a range of conditions, including variable pH.

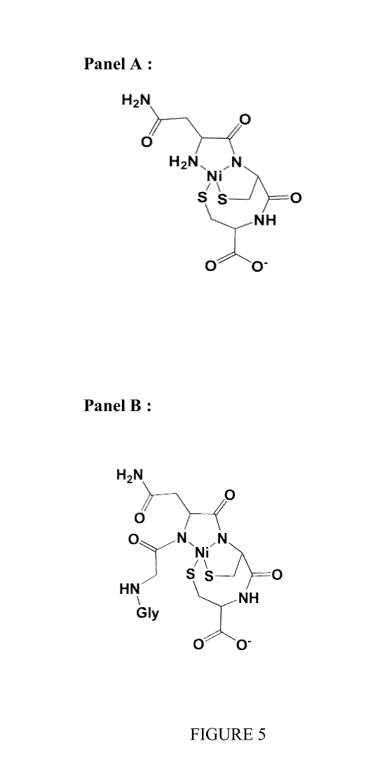

Once incorporated, the metal is believed to be held in a square planar, 2N:2S geometry. The two sulfur atoms from the Cys residues and two backbone nitrogen atoms coordinate the metal. The NCC tripeptide binds using the N-terminal amine, yet metal is also readily incorporated when the NCC sequence is placed within longer peptide sequences. FIG. 5 panel A shows a possible binding arrangement for a NCC sequence with nickel. Panel B illustrates a possible binding arrangement for a GGNCC (SEQ ID NO.: 1) sequence to nickel.

### Example 2

### Other Metals

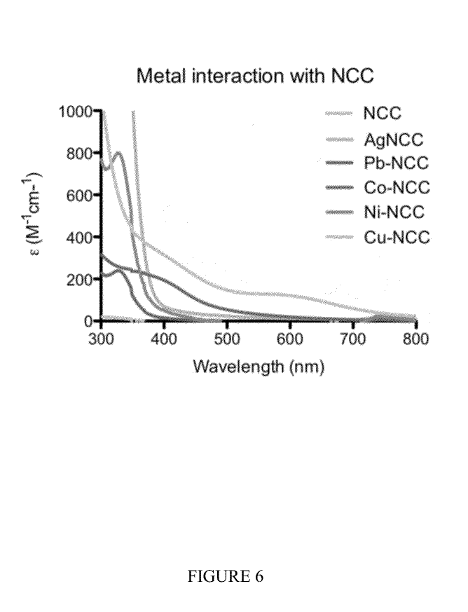

Along with nickel and platinum, several other soft metals were successfully been incorporated into the tag, including silver, lead, cobalt, and copper (FIG. 6) by the methods of Example 1.

### Example 3

### pH Dependent Release of Metal

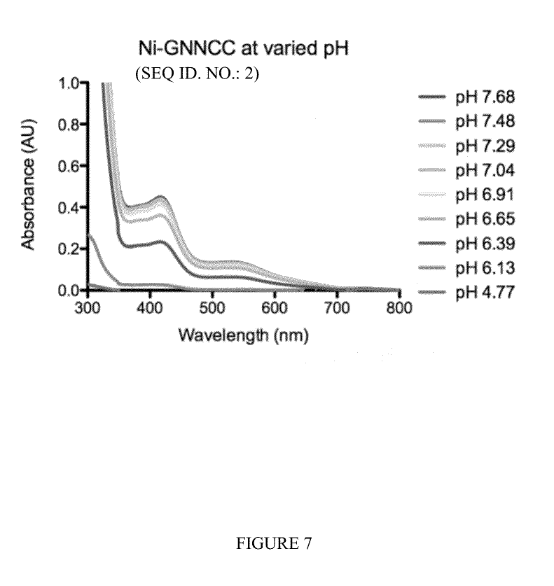

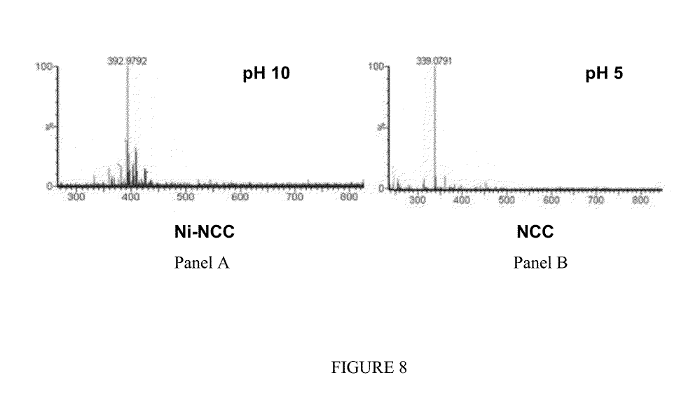

The metal-MAP complex is extremely stable; even with exposure to light and oxygen, the absorption data for Ni-NCC remained unchanged for 90 days, indicating that the complex remains intact. With exposure to common chelators, reducing agents, denaturants, or heat, the complex remains intact; however, acidification of the metal-MAP complex resulted in a change in the absorbance spectrum and mass spectrometry. FIG. 7 demonstrates that a loss of intensity in the absorbance profile corresponds to release of metal from the GNNCC (SEQ ID NO: 2) peptide. FIG. 8 demonstrates the metal release profile of a Ni-NCC complex at pH 10 and pH 5 by mass spectroscopy.

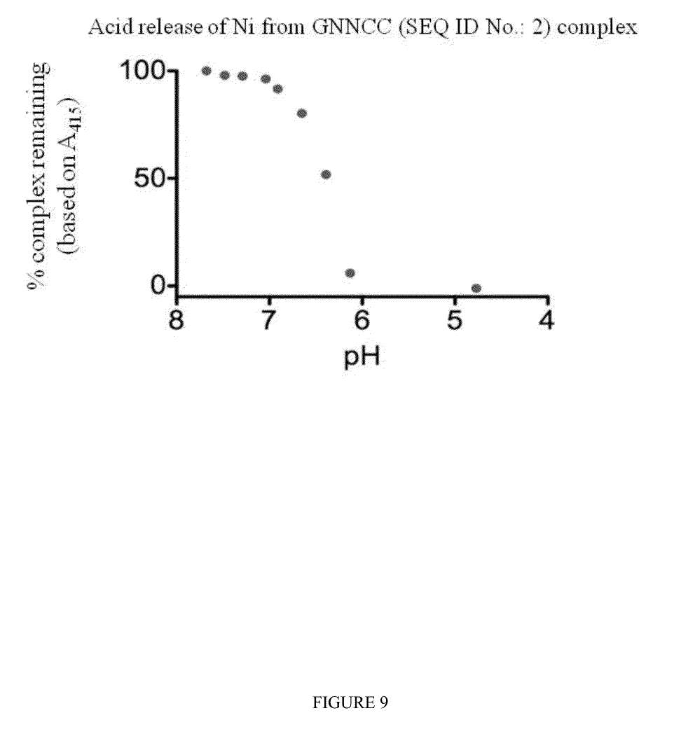

The release of metal from the peptide occurred just below physiological pH and occurred immediately upon exposure to the lower pH condition. The loss of absorbance intensity corresponds to loss of metal from the complex, as the absorption features that are observed for Ni-MAP are due to ligand to metal charge transfer (LMCT) bands and d to d transitions. By monitoring the intensity of one specific peak (415 nm for the pentapteptide) with decreasing pH, the percent of the complex remaining at each pH unit was calculated and plotted (FIG. 9).

### Example 4

### Complex in Presence of Ammonium Chloride

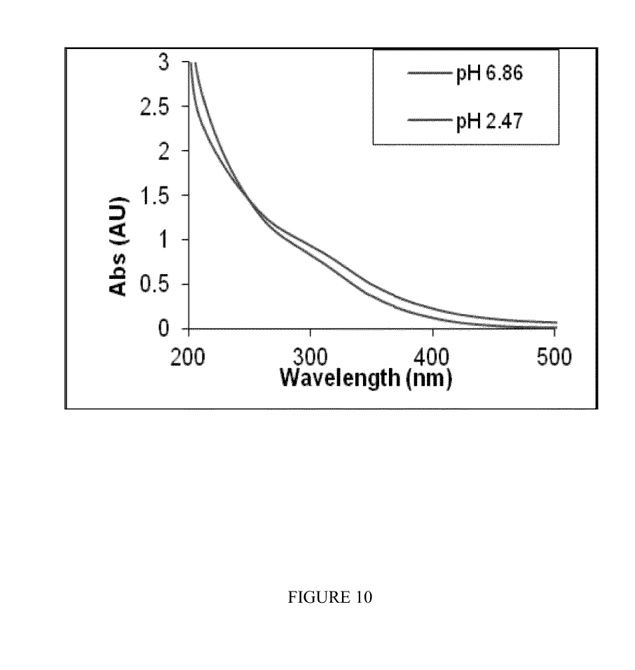

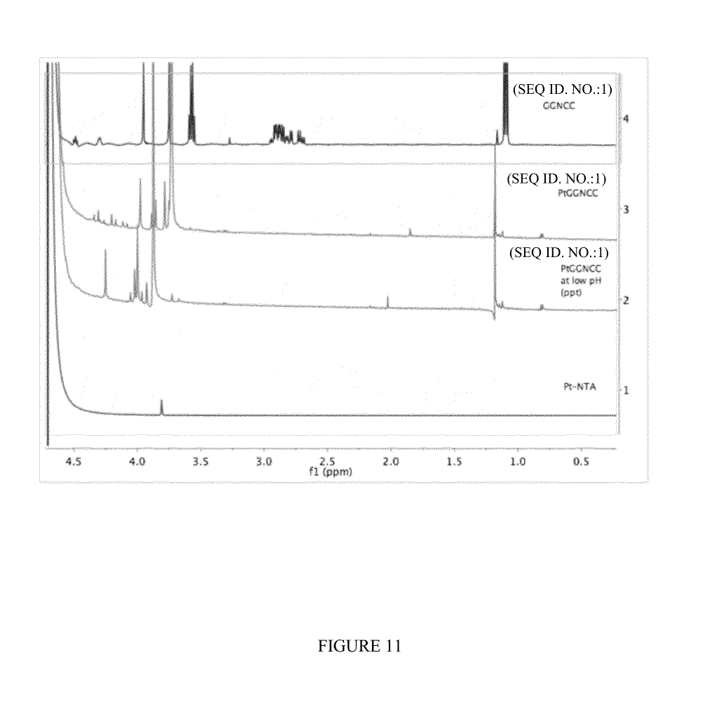

Upon lowering the pH of a Pt-peptide complex, precipitation was immediately observed, indicating that a change took place and that the metal was released. Because Pt is bound to ammonia and chloride ligands in the common inorganic complex cisplatin, the release experiment was also performed in the presence of excess ammonium chloride. While some precipitation occurred, a difference in the absorption profile was still observed. A clear isosbestic point was observed at approximately 250 nm, suggesting the conversion from one species to another (FIG. 10). Additionally, ¹H NMR of the GGNCC (SEQ ID NO.:1), and Pt-GGNCC (SEQ ID NO.:1) at low pH were all performed. Precipitation occurred in the Pt-GGNCC (SEQ ID NO.:1) sample at low pH, but some species remained in solution at this pH, as peaks were still present in the NMR (FIG. 11).

### Example 5

### Release of Metal with Chance in pH

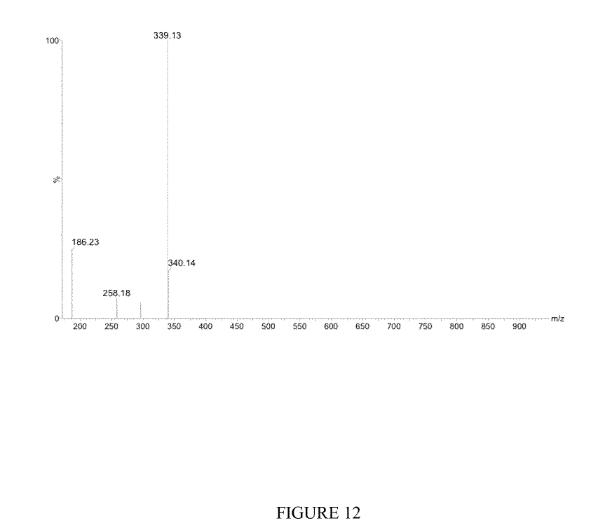

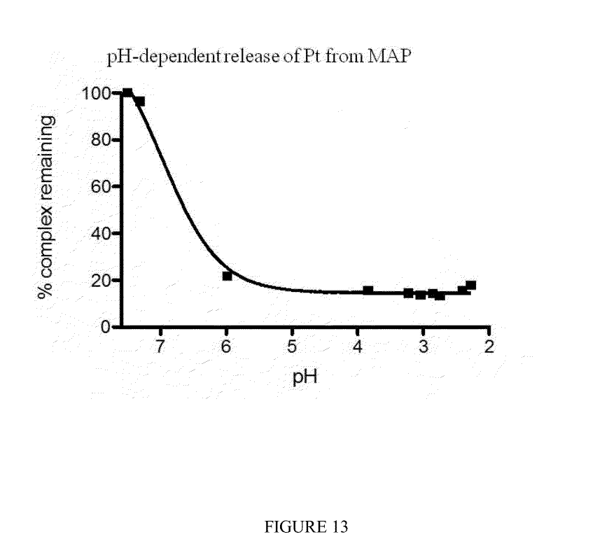

MS data was obtained at low pH when solvent (DMSO) was added to the peptide-metal complex before dropping the pH, to keep some of the species that result from metal release soluble. Although a small amount of a yellow precipitate formed, the peptide returned to the apo state and remained in solution as determined by MS (FIG. 12). By adding DMSO to the solution, a similar release experiment was performed using absorption spectroscopy. As with nickel, the release of metal from the peptide occurred just below physiological pH and occurred immediately upon exposure to the lower pH condition. The loss of absorbance intensity corresponded to loss of metal from the complex. The loss of intensity was monitored at 315 nm, as this is a significant, yet unique, feature in the Pt-NCC spectral profile, and pH dependent release curve was prepared (FIG. 13).

### Example 6

### Release of Cobalt

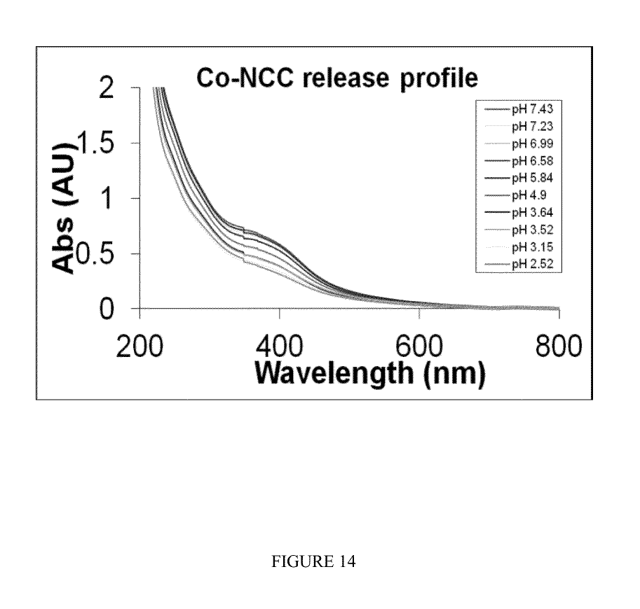

FIG. 14 shows the release of cobalt from NCC using absorbance experiments described above.

### Example 7

### Modulation of X Position in XCC Peptide Influences pH-Dependent Release of Metal

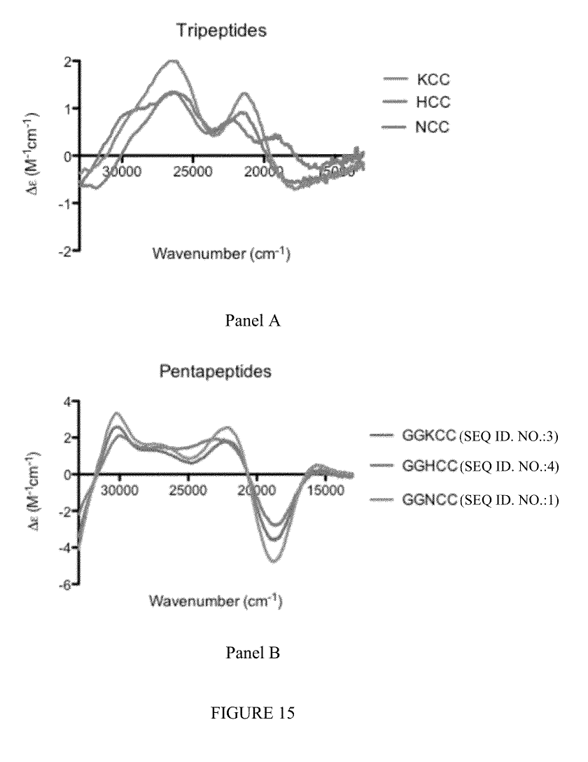

The peptide complexes Ni-HCC, Ni-KCC, Ni-GGHCC (SEQ ID NO.:4), and Ni-GGKCC (SEQ ID NO.:3) were prepared. CD spectra showed that the presence of basic residues does not alter the primary coordination of metal (FIG. 15), as the spectra have similar features. FIG. 15 panel A illustrates the absorbance spectra of exemplary tripeptides. FIG. 15 panel B illustrates the absorbance spectra of exemplary pentapeptides. The small deviation in the spectra of the pentapeptides likely resulted from transient differences in the orientation and axial interaction of the side chain moiety above the plane of the coordinated metal. For example, the imidazolium nitrogen of histidine in the X position can ligate the metal axial to the square planar 2N:2S structure, resulting in square pyramidal geometry. Lysine, which has a positively charged side chain, can interact with the negatively charged square planar 2N:2S metal center.

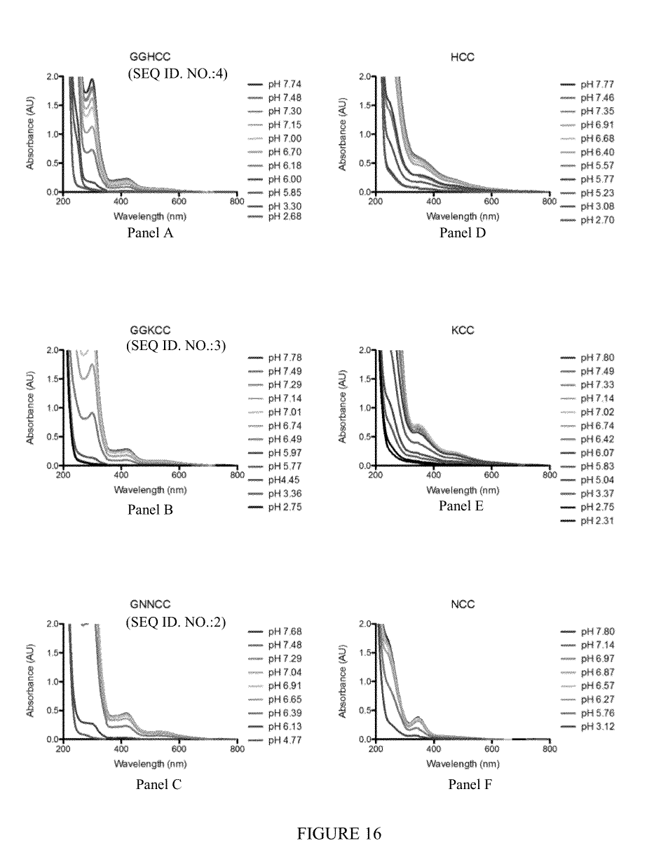

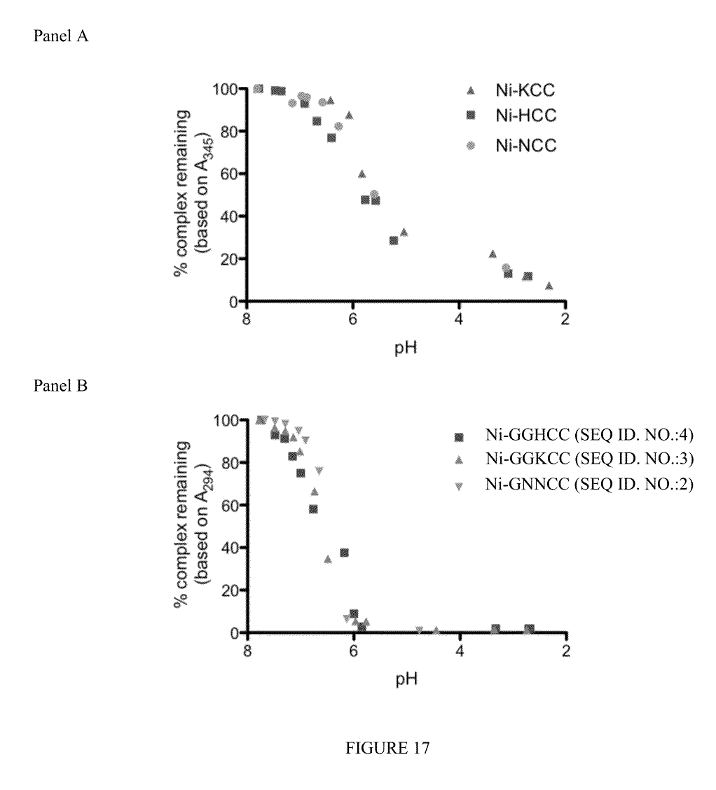

pH release experiments were then performed (FIG. 16), and release curves were prepared for each of the six peptides (FIG. 17). FIG. 16 panel A shows the pH-dependent metal release of Ni-GGHCC (SEQ ID NO. 4) complexes. Panel B shows the pH-dependent metal release of Ni-GGKCC (SEQ ID NO. 3) complexes. Panel C shows the pH-dependent metal release of Ni-GNNCC (SEQ ID NO. 2) complexes. Panel D shows the pH-dependent metal release of Ni-HCC complexes. Panel E shows the pH-dependent metal release of Ni-KCC complexes. Panel F shows the pH-dependent metal release of Ni-NCC complexes. FIG. 17 panel A shows the pH dependent release profile for Ni-tripeptide complexes. FIG. 17 panel B shows the pH dependent release profile for Ni-pentapeptide complexes.

The data illustrate that modulating the ligand environment near the metal center, by modifying the peptide, impacted the pH at which metal was released from the peptide. The pentapeptide system fully released the metal by dropping to pH 6.0. The release of the metal from the peptide system at pH values that are attainable in the desired places within physiological systems (i.e. the slightly acidic environment present in cancer cells) supports placement of the MAP tag within longer protein sequences.

As seen in FIGS. 16 and 17, changing the asparagine residue to either a histidine or a lysine residue changed the release profile. These data indicate that the identity of the residue in the X position modulates the pH at which metal is released.

### Example 8

### Placement MAP in a Protein Sequence Modulated Metal Release

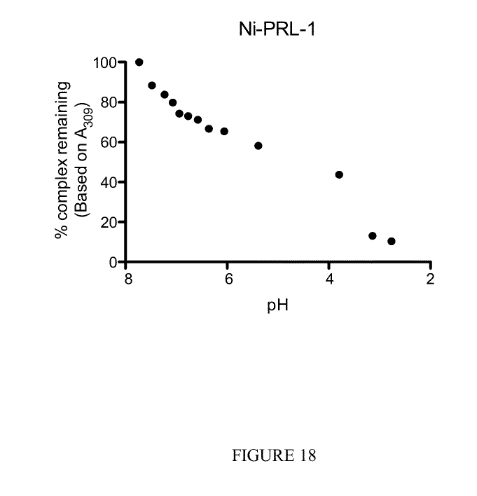

Placement of MAP in a longer sequence modulated metal release due to the change from a mixed amine-amide to a bis-amide nitrogen system. Modulating the amino acid sequence modulated the release (FIG. 17). The experiment to observe nickel release from the MAP tag within the protein phosphatase of regenerating liver 1 (PRL-1) was performed by sequentially dropping the pH of a Ni-PRL-1 sample and monitoring the resulting absorption spectra. As with the Ni-MAP peptides, the loss of absorbance intensity corresponds to loss of metal from the MAP tag. The release of metal from the peptide occurred just below physiological pH and occurred immediately upon exposure to the lower pH condition (FIG. 18); however, in the case of Ni-PRL-1, the release was not complete until the pH of the bulk solution was much more acidic. Since the sequence near to the MAP tag was RRNNCCIQ (SEQ ID NO.: 5), which contains several basic residues, the neighboring residues modulated the release by buffering locally, keeping the local pH at the site of the MAP tag higher than the pH of the bulk solution.

### Example 9

### Addition of the MAP Tag to Epidermal Growth Factor

The epidermal growth factor receptor (EGFR) is expressed on the surface of head and neck cancer cells. Epidermal growth factor (EGF) is a small, 53 amino acid protein that binds to EGFR. The sequence GNCCG (SEQ ID NO.:6) was added to the N-terminal end of EGF to provide a complex, preventing undesired obstruction of the protein fold by the addition of the tag. The complex was expressed in E. coli, purified, and in some constructs refolded. Incorporation of the GNCCG (SEQ ID NO.:6) tag did not affect expression, purification, or stability of EGF. Pt was incorporated into the complex, with chelated platinum, and metal incorporation was validated using atomic absorption spectroscopy (AA) as well as an observation of a shift in the peptide backbone absorption to lower wavelength.

### Embodiments

The following non-limiting embodiments provide illustrative examples of the invention, but do not limit the scope of the invention.

Embodiment 1. A pharmaceutical composition comprising: i) a metal-binding peptide, wherein the metal-binding peptide comprises a sequence Z¹—XZ³C₁Z⁴C₂—Z², wherein X is any natural or non-natural amino acid or amino acid analogue, and wherein C₁ and C₂ are each individually chosen from a cysteine and a sulfur-containing alpha or beta amino acid, and wherein Z¹, Z², Z³, and Z⁴ are each individually a sequence of 1-5 residues, or absent, wherein each residue is independently a natural or non-natural amino acid or analogue thereof; ii) a metal bound to the metal-binding peptide; and iii) a pharmaceutically-acceptable excipient, wherein the composition is a unit dosage form.

Embodiment 2. The pharmaceutical composition of Embodiment 1, wherein the metal-binding peptide binds the metal to yield a concentration of peptide with bound metal and a concentration of peptide without bound metal and a ratio of the concentrations, wherein the ratio is at least two times greater at a pH above 7 than the ratio at a pH below 6 for a constant concentration of metal-binding peptide and metal.

Embodiment 3. The pharmaceutical composition of any one of Embodiments 1 and 2, wherein the ratio of the concentrations is about 2 to about 1.

Embodiment 4. The pharmaceutical composition of Embodiment 2, wherein the ratio is at least five times greater at a pH above 7 than the ratio at a pH below 6.

Embodiment 5. The pharmaceutical composition of Embodiment 2, wherein the ratio of the concentrations is at least five times greater at a pH between 7 and 8 than the ratio at a pH between 4 and 6.

Embodiment 6. The pharmaceutical composition of Embodiment 2, wherein the metal-binding peptide comprises at least 20 amino acid residues.

Embodiment 7. The pharmaceutical composition of any one of Embodiments 1-6, wherein X is a basic amino acid.

Embodiment 8. The pharmaceutical composition of any one of Embodiments 1-7, wherein at least one of C₁ and C₂ is cysteine.

Embodiment 9. The pharmaceutical composition of any one of Embodiments 1-8, wherein at least one of Z¹ and Z² includes a basic amino acid adjacent to X or C₂.

Embodiment 10. The pharmaceutical composition of any one of Embodiments 1-9, wherein at least one of Z³ and Z⁴ includes a basic amino acid adjacent to X, C₁, or C₂.

Embodiment 11. The pharmaceutical composition of any one of Embodiments 1-8, wherein Z², Z³, and Z⁴ are absent, wherein Z¹ is any natural or non-natural amino acid or sequence of natural or non-natural amino acids.

Embodiment 12. The pharmaceutical composition of Embodiment 11, wherein a basic amino acid of Z¹ is adjacent to X.

Embodiment 13. The pharmaceutical composition any one of Embodiments 1-8, wherein Z¹ and Z² are absent.

Embodiment 14. The pharmaceutical composition of any one of Embodiments 1-8, wherein Z¹, Z², Z³, and Z⁴ are absent.

Embodiment 15. The pharmaceutical composition of any one of Embodiments 1-8, wherein Z³ and Z⁴ are absent.

Embodiment 16. The pharmaceutical composition of any one of Embodiments 1-15, wherein the metal-binding peptide is linked to an antibody.

Embodiment 17. The pharmaceutical composition of Embodiment 16, wherein the metal-binding peptide is linked to the antibody through an amide bond.

Embodiment 18. The pharmaceutical composition of any one of Embodiments 1-17, wherein the metal is platinum.

Embodiment 19. The pharmaceutical composition of any one of Embodiments 1-18, wherein the pharmaceutically-acceptable excipient is a phosphate buffer.

Embodiment 20. The pharmaceutical composition of any one of Embodiments 1-19, wherein the unit dosage form provides a therapeutically-effective amount of the metal bound to the metal-binding peptide to a subject, after administration to the subject.

Embodiments 21. The pharmaceutical composition of Embodiment 20, wherein the therapeutically-effective amount of the metal bound to the metal-binding peptide is from about 1 mg to about 100 mg.

Embodiment 22. The pharmaceutical composition of Embodiment 20, wherein the therapeutically-effective amount of the metal bound to the metal-binding peptide is from about 100 mg to about 5000 mg.

Embodiment 23. The pharmaceutical composition of Embodiment 20, wherein the subject is a human.

Embodiment 24. A method of treating cancer, the method comprising administering to a subject in need or want thereof a therapeutically-effective amount of a composition comprising: i) a metal-binding peptide, wherein the metal-binding peptide comprises a sequence Z¹—XZ³C₁Z⁴C₂—Z², wherein X is any natural or non-natural amino acid or amino acid analogue, and wherein C₁ and C₂ are each individually chosen from a cysteine and a sulfur-containing alpha or beta amino acid, and wherein Z¹, Z², Z³, and Z⁴ are each individually a sequence of 1-5 residues, or absent, wherein each residue is independently a natural or non-natural amino acid or analogue thereof; ii) a metal bound to the metal-binding peptide; and iii) a pharmaceutically-acceptable excipient, wherein the composition is a unit dosage form.

Embodiment 25. The method of Embodiment 24, wherein the metal-binding peptide binds the metal to yield a concentration of peptide with bound metal and a concentration of peptide without bound metal and a ratio of the concentrations, wherein the ratio is at least two times greater at a pH above 7 than the ratio at a pH below 6 for a constant concentration of metal-binding peptide and metal.

Embodiment 26. The method of Embodiment 25, wherein the ratio of the concentrations is about 2 to about 1.

Embodiment 27. The method of Embodiment 25, wherein the ratio is at least five times greater at a pH above 7 than the ratio at a pH below 6.

Embodiment 28. The method of Embodiment 25, wherein the ratio of the concentrations is at least five times greater at a pH between 7 and 8 than the ratio at a pH between 4 and 6.

Embodiment 29. The method of any one of Embodiments 24-28, wherein the metal-binding peptide comprises at least 20 amino acid residues.

Embodiment 30. The method of any one of Embodiments 24-29, wherein X is a basic amino acid.

Embodiment 31. The method of any one of Embodiments 24-30, wherein at least one of C₁ and C₂ is cysteine.

Embodiment 32. The method of any one of Embodiments 24-31, wherein at least one of Z¹ and Z² includes a basic amino acid adjacent to X or C₂.

Embodiment 33. The method of any one of Embodiments 24-32, wherein at least one of Z³ and Z⁴ includes a basic amino acid adjacent to X, C₁, or C₂.

Embodiment 34. The method of any one of Embodiments 24-31, wherein Z², Z³, and Z⁴ are absent, wherein Z¹ is any natural or non-natural amino acid or sequence of natural or non-natural amino acids.

Embodiment 35. The method of Embodiment 34, wherein a basic amino acid of Z¹ is adjacent to X.

Embodiment 36. The method of any one of Embodiments 24-31, wherein Z¹ and Z² are absent.

Embodiment 37. The method of any one of Embodiments 24-31, wherein Z¹, Z², Z³, and Z⁴ are absent.

Embodiment 38. The method of any one of Embodiments 24-31, wherein Z³ and Z⁴ are absent.

Embodiment 39. The method of any one of Embodiments 24-38, wherein the metal-binding peptide is linked to an antibody.

Embodiment 40. The method of Embodiment 39, wherein the metal-binding peptide is linked to the antibody through an amide bond.

Embodiment 41. The method of any one of Embodiments 24-40, wherein the metal is platinum.

Embodiment 42. The method of any one of Embodiments 32-41, wherein the pharmaceutically-acceptable excipient is a phosphate buffer.

Embodiment 43. The method of any one of Embodiments 24-42, wherein the concentration of peptide with bound metal provides a therapeutically-effective amount of peptide with bound metal to a subject, after administration to the subject.

Embodiment 44. The method of any one of Embodiments 24-43, wherein the therapeutically-effective amount of peptide with bound metal is from about 1 mg to about 500 mg.

Embodiment 45. The method of any one of Embodiments 24-43, wherein the therapeutically-effective amount of peptide with bound metal is from about 100 mg to about 5000 mg.

Embodiment 46. The method of any one of Embodiments 24-45, wherein the subject is a human.

Embodiment 47. A method of providing a metal to a subject, the method comprising administering to the subject: i) a metal-binding peptide, wherein the metal-binding peptide comprises a sequence Z¹—XZ³C₁Z⁴C₂—Z², wherein X is any natural or non-natural amino acid or amino acid analog, and wherein C₁ and C₂ are each individually chosen from a cysteine and a sulfur-containing alpha or beta amino acid, and wherein Z¹, Z², Z³, and Z⁴ are each individually a sequence of 1-5 residues, or absent, wherein each residue is independently a natural or non-natural amino acid or analogue thereof; and ii) the metal, wherein the metal is bound to the metal-binding peptide, wherein the metal-binding peptide releases the metal in a physiological environment, wherein the release is associated with a decrease in pH.

Embodiment 48. The method of Embodiment 47, wherein the metal is released at a pH below 7.

Embodiment 49. The method of any one of Embodiments 47 and 48, wherein the metal is released at a pH of 6 or below.

Embodiment 50. The method of any one of Embodiments 47-49, wherein the metal is released at a pH of 5 or below.

Embodiment 51. The method of any one of Embodiments 47-50, wherein the physiological environment is a tumor cell.

Embodiment 52. The method of any one of Embodiments 47-51, wherein the physiological environment is a cancer cell.

Embodiment 53. The method of any one of Embodiments 47-52, wherein X is a basic amino acid.

Embodiment 54. The method of any one of Embodiments 47-53, wherein at least one of C₁ and C₂ is cysteine.

Embodiment 55. The method of any one of Embodiments 47-54, wherein at least one of Z¹ and Z² includes a basic amino acid adjacent to X or C₂.

Embodiment 56. The method of any one of Embodiments 47-55, wherein at least one of Z³ and Z⁴ includes a basic amino acid adjacent to X, C₁, or C₂.

Embodiment 57. The method of any one of Embodiments 47-56, wherein Z², Z³, and Z⁴ are absent, wherein Z¹ is any natural or non-natural amino acid or sequence of natural or non-natural amino acids.

Embodiment 58. The method of Embodiment 57, wherein a basic amino acid of Z¹ is adjacent to X.

Embodiment 59. The method of any one of Embodiments 47-54, wherein Z¹ and Z² are absent.

Embodiment 60. The method of any one of Embodiments 47-54, wherein Z¹, Z², Z³, and Z⁴ are absent.

Embodiment 61. The method of any one of Embodiments 47-54, wherein Z³ and Z⁴ are absent.

Embodiment 62. The method of any one of Embodiments 47-61, wherein the metal-binding peptide is linked to an antibody.

Embodiment 63. The method of Embodiment 62, wherein the metal-binding peptide is linked to the antibody through an amide bond.

Embodiment 64. The method of any one of Embodiments 47-63, wherein the metal is platinum.

Embodiment 65. The method of any one of Embodiments 47-64, wherein the administering is intravenous.

Embodiment 66. The method of any one of Embodiments 47-65, wherein the subject is a human.

Embodiment 67. A pharmaceutical composition comprising: i) a metal-binding peptide, wherein the metal-binding peptide comprises a sequence Z¹—XZ³C₁Z⁴C₂—Z², wherein X is any natural or non-natural amino acid or amino acid analogue, and wherein C₁ and C₂ are each individually chosen from a cysteine and a sulfur-containing alpha or beta amino acid, and wherein Z¹, Z², Z³, and Z⁴ are each individually a sequence of 1-5 residues, or absent, wherein each residue is independently a natural or non-natural amino acid or analogue thereof; ii) a metal bound to the metal-binding peptide; and iii) a pharmaceutically-acceptable excipient, wherein the composition is a unit dosage form.

Embodiment 68. The pharmaceutical composition of Embodiment 67, wherein the metal is a radioisotope.

Embodiment 69. The pharmaceutical composition of Embodiment 68, wherein the radioisotope is lutetium-177.

Embodiment 70. The pharmaceutical composition of Embodiment 68, wherein the radioisotope is terbium-161.

Embodiment 71. The pharmaceutical composition of Embodiment 68, wherein the radioisotope is an isotope of platinum.

Embodiment 72. The pharmaceutical composition of Embodiment 68, wherein the radioisotope is of a transition metal.

Embodiment 73. The pharmaceutical composition of Embodiment 68, wherein the radioisotope is of a main group metal.

Embodiment 74. The pharmaceutical composition of Embodiment 68, wherein the radioisotope is of a lanthanide metal.

Embodiment 75. The pharmaceutical composition of Embodiment 68, wherein the radioisotope is of an actinide metal.

Embodiment 76. A method of treating cancer, the method comprising administering to a subject in need or want thereof a therapeutically-effective amount of a composition comprising: i) a metal-binding peptide, wherein the metal-binding peptide comprises a sequence Z¹—XZ³C₁Z⁴C₂—Z², wherein X is any natural or non-natural amino acid or amino acid analogue, and wherein C₁ and C₂ are each individually chosen from a cysteine and a sulfur-containing alpha or beta amino acid, and wherein Z¹, Z², Z³, and Z⁴ are each individually a sequence of 1-5 residues, or absent, wherein each residue is independently a natural or non-natural amino acid or analogue thereof; ii) a metal bound to the metal-binding peptide; and iii) a pharmaceutically-acceptable excipient, wherein the composition is a unit dosage form.

Embodiment 77. The pharmaceutical composition of Embodiment 76, wherein the metal is a radioisotope.

Embodiment 78. The pharmaceutical composition of Embodiment 77, wherein the radioisotope is lutetium-177.

Embodiment 79. The pharmaceutical composition of Embodiment 77, wherein the radioisotope is terbium-161.

Embodiment 80. The pharmaceutical composition of Embodiment 77, wherein the radioisotope is an isotope of platinum.

Embodiment 81. The pharmaceutical composition of Embodiment 77, wherein the radioisotope is of a transition metal.

Embodiment 82. The pharmaceutical composition of Embodiment 77, wherein the radioisotope is of a main group metal.

Embodiment 83. The pharmaceutical composition of Embodiment 77, wherein the radioisotope is of a lanthanide metal.

Embodiment 84. The pharmaceutical composition of Embodiment 77, wherein the radioisotope is of an actinide metal.

Embodiment 85. A method of providing a metal to a subject, the method comprising administering to the subject: i) a metal-binding peptide, wherein the metal-binding peptide comprises a sequence Z¹—XZ³C₁Z⁴C₂—Z², wherein X is any natural or non-natural amino acid or amino acid analog, and wherein C₁ and C₂ are each individually chosen from a cysteine and a sulfur-containing alpha or beta amino acid, and wherein Z¹, Z², Z³, and Z⁴ are each individually a sequence of 1-5 residues, or absent, wherein each residue is independently a natural or non-natural amino acid or analogue thereof; and ii) the metal, wherein the metal is bound to the metal-binding peptide, wherein the metal-binding peptide releases the metal in a physiological environment, wherein the release is associated with a decrease in pH.

Embodiment 86. The pharmaceutical composition of Embodiments 85, wherein the metal is a radioisotope.

Embodiment 87. The pharmaceutical composition of Embodiment 86, wherein the radioisotope is lutetium-177.

Embodiment 88. The pharmaceutical composition of Embodiment 86, wherein the radioisotope is terbium-161.

Embodiment 89. The pharmaceutical composition of Embodiment 86, wherein the radioisotope is an isotope of platinum.

Embodiment 90. The pharmaceutical composition of Embodiment 86, wherein the radioisotope is of a transition metal.

Embodiment 91. The pharmaceutical composition of Embodiment 86, wherein the radioisotope is of a main group metal.

Embodiment 92. The pharmaceutical composition of Embodiment 86, wherein the radioisotope is of a lanthanide metal.

Embodiment 93. The pharmaceutical composition of Embodiment 86, wherein the radioisotope is of an actinide metal.

What is claimed is:

1. A pharmaceutical composition comprising:
i) a metal-binding peptide, wherein the metal-binding peptide comprises a sequence Z¹—XZ³C₁Z⁴C₂—Z², wherein X is any natural or non-natural amino acid or amino acid analogue, and wherein C₁ and C₂ are each individually chosen from a cysteine and a sulfur-containing alpha or beta amino acid, and wherein Z¹, Z², Z³, and Z⁴ are each individually a sequence of 1-5 residues, or absent, wherein each residue is independently a natural or non-natural amino acid or analogue thereof;
ii) a metal bound to the metal-binding peptide; and
iii) a pharmaceutically-acceptable excipient,
wherein the composition is a unit dosage form.

2. The pharmaceutical composition of claim 1, wherein the metal-binding peptide binds the metal to yield a concentration of peptide with bound metal and a concentration of peptide without bound metal and a ratio of the concentrations, wherein the ratio is at least two times greater at a pH above 7 than the ratio at a pH below 6 for a constant concentration of metal-binding peptide and metal.

3. The pharmaceutical composition of claim 2, wherein the ratio of the concentrations is about 2 to about 1.

4. The pharmaceutical composition of claim 2, wherein the ratio is at least five times greater at a pH above 7 than the ratio at a pH below 6.

5. The pharmaceutical composition of claim 2, wherein the ratio of the concentrations is at least five times greater at a pH between 7 and 8 than the ratio at a pH between 4 and 6.

6. The pharmaceutical composition of claim 1, wherein the metal-binding peptide comprises at least 20 amino acid residues.

7. The pharmaceutical composition of claim 1, wherein X is a basic amino acid.

8. The pharmaceutical composition of claim 1, wherein at least one of C₁ and C₂ is cysteine.

9. The pharmaceutical composition of claim 1, wherein at least one of Z¹ and Z² includes a basic amino acid adjacent to X or C₂.

10. The pharmaceutical composition of claim 1, wherein at least one of Z³ and Z⁴ includes a basic amino acid adjacent to X, C₁, or C₂.

11. The pharmaceutical composition of claim 1, wherein Z², Z³, and Z⁴ are absent, wherein Z¹ is any natural or non-natural amino acid or sequence of natural or non-natural amino acids.

12. The pharmaceutical composition of claim 11, wherein a basic amino acid of Z¹ is adjacent to X.

13. The pharmaceutical composition of claim 1, wherein Z¹ and Z² are absent.

14. The pharmaceutical composition of claim 1, wherein Z¹, Z², Z³, and Z⁴ are absent.

15. The pharmaceutical composition of claim 1, wherein Z³ and Z⁴ are absent.

16. The pharmaceutical composition of claim 1, wherein the metal-binding peptide is linked to an antibody.

17. The pharmaceutical composition of claim 16, wherein the metal-binding peptide is linked to the antibody through an amide bond.

18. The pharmaceutical composition of claim 1, wherein the metal is platinum.

19. The pharmaceutical composition of claim 1, wherein the pharmaceutically-acceptable excipient is a phosphate buffer.

20. The pharmaceutical composition of claim 1, wherein the unit dosage form provides a therapeutically-effective amount of the metal bound to the metal-binding peptide to a subject, after administration to the subject.

21. The pharmaceutical composition of claim 20, wherein the therapeutically-effective amount of the metal bound to the metal-binding peptide is from about 1 mg to about 100 mg.

22. The pharmaceutical composition of claim 20, wherein the therapeutically-effective amount of the metal bound to the metal-binding peptide is from about 100 mg to about 5000 mg.

23. The pharmaceutical composition of claim 20, wherein the subject is a human.

24. A method of treating cancer, the method comprising administering to a subject in need or want thereof a therapeutically-effective amount of a composition comprising:
i) a metal-binding peptide, wherein the metal-binding peptide comprises a sequence Z¹—XZ³C₁Z⁴C₂—Z², wherein X is any natural or non-natural amino acid or amino acid analogue, and wherein C₁ and C₂ are each individually chosen from a cysteine and a sulfur-containing alpha or beta amino acid, and wherein Z¹, Z², Z³, and Z⁴ are each individually a sequence of 1-5 residues, or absent, wherein each residue is independently a natural or non-natural amino acid or analogue thereof;
ii) a metal bound to the metal-binding peptide; and
iii) a pharmaceutically-acceptable excipient,
wherein the composition is a unit dosage form.

25. The method of claim 24, wherein the metal-binding peptide binds the metal to yield a concentration of peptide with bound metal and a concentration of peptide without bound metal and a ratio of the concentrations, wherein the ratio is at least two times greater at a pH above 7 than the ratio at a pH below 6 for a constant concentration of metal-binding peptide and metal.

26. The method of claim 25, wherein the ratio of the concentrations is about 2 to about 1.

27. The method of claim 25, wherein the ratio is at least five times greater at a pH above 7 than the ratio at a pH below 6.

28. The method of claim 25, wherein the ratio of the concentrations is at least five times greater at a pH between 7 and 8 than the ratio at a pH between 4 and 6.

29. The method of claim 24, wherein the metal-binding peptide comprises at least 20 amino acid residues.

30. The method of claim 24, wherein X is a basic amino acid.

31. The method of claim 24, wherein at least one of C₁ and C₂ is cysteine.

32. The method of claim 24, wherein at least one of Z¹ and Z² includes a basic amino acid adjacent to X or C₂.

33. The method of claim 24, wherein at least one of Z³ and Z⁴ includes a basic amino acid adjacent to X, C₁, or C₂.

34. The method of claim 24, wherein Z², Z³, and Z⁴ are absent, wherein Z¹ is any natural or non-natural amino acid or sequence of natural or non-natural amino acids.

35. The method of claim 34, wherein a basic amino acid of Z¹ is adjacent to X.

36. The method of claim 24, wherein Z¹ and Z² are absent.

37. The method of claim 24, wherein Z¹, Z², Z³, and Z⁴ are absent.

38. The method of claim 24, wherein Z³ and Z⁴ are absent.

39. The method of claim 24, wherein the metal-binding peptide is linked to an antibody.

40. The method of claim 39, wherein the metal-binding peptide is linked to the antibody through an amide bond.

41. The method of claim 24, wherein the metal is platinum.

42. The method of claim 24, wherein the pharmaceutically-acceptable excipient is a phosphate buffer.

43. The method of claim 24, wherein the administering is intravenous.

44. The method of claim 24, wherein the therapeutically-effective amount of peptide with bound metal is from about 1 mg to about 100 mg.

45. The method of claim 24, wherein the therapeutically-effective amount of peptide with bound metal is from about 100 mg to about 5000 mg.

46. The method of claim 24, wherein the subject is a human.

47. A method of providing a metal to a subject in need or want thereof, the method comprising administering to the subject:
i) a metal-binding peptide, wherein the metal-binding peptide comprises a sequence Z¹—XZ³C₁Z⁴C₂—Z², wherein X is any natural or non-natural amino acid or amino acid analog, and wherein C₁ and C₂ are each individually chosen from a cysteine and a sulfur-containing alpha or beta amino acid, and wherein Z¹, Z², Z³, and Z⁴ are each individually a sequence of 1-5 residues, or absent, wherein each residue is independently a natural or non-natural amino acid or analogue thereof; and
ii) the metal, wherein the metal is bound to the metal-binding peptide,
wherein the metal-binding peptide releases the metal in a physiological environment, wherein the release is associated with a decrease in pH.

48. The method of claim 47, wherein the metal is released at a pH below 7.

49. The method of claim 47, wherein the metal is released at a pH of 6 or below.

50. The method of claim 47, wherein the metal is released at a pH of 5 or below.

51. The method of claim 47, wherein the physiological environment is a tumor cell.

52. The method of claim 47, wherein the physiological environment is a cancer cell.

53. The method of claim 47, wherein X is a basic amino acid.

54. The method of claim 47, wherein at least one of C₁ and C₂ is cysteine.

55. The method of claim 47, wherein at least one of Z¹ and Z² includes a basic amino acid adjacent to X or C₂.

56. The method of claim 47, wherein at least one of Z³ and Z⁴ includes a basic amino acid adjacent to X, C₁, or C₂.

57. The method of claim 47, wherein Z², Z³, and Z⁴ are absent, wherein Z¹ is any natural or non-natural amino acid or sequence of natural or non-natural amino acids.

58. The method of claim 57, wherein a basic amino acid of Z¹ is adjacent to X.

59. The method of claim 47, wherein Z¹ and Z² are absent.

60. The method of claim 47, wherein Z¹, Z², Z³, and Z⁴ are absent.

61. The method of claim 47, wherein Z³ and Z⁴ are absent.

62. The method of claim 47, wherein the metal-binding peptide is linked to an antibody.

63. The method of claim 62, wherein the metal-binding peptide is linked to the antibody through an amide bond.

64. The method of claim 47, wherein the metal is platinum.

65. The method of claim 47, wherein the administering is intravenous

66. The method of claim 47, wherein the subject is a human.
